(19) United States
(12) Patent Application Publication
(10) Pub. No.: US 2024/0002770 A1
(43) Pub. Date: Jan. 4, 2024

(54) SYSTEMS, APPARATUS, AND METHODS FOR CELL CULTURE

(71) Applicants: Genentech, Inc., South San Francisco, CA (US); Hoffmann-La Roche Inc., Little Falls, NJ (US)

(72) Inventors: Edward Hon Man CHAN, San Francisco, CA (US); Rigzen Pratap Singh AULAKH, Brisbane, CA (US); Dominik MARKS, Iffeldorf (DE); Christian SCHANTZ, Penzberg (DE)

(21) Appl. No.: 18/344,589

(22) Filed: Jun. 29, 2023

Related U.S. Application Data
(60) Provisional application No. 63/357,593, filed on Jun. 30, 2022.

(51) Int. Cl. C12M 1/00; C12M 1/34

(52) U.S. Cl. CPC C12M 29/04; C12M 41/40; C12M 41/46; C12M 29/10; C12M 47/02

(57) ABSTRACT

Embodiments described herein generally relate to assemblies, systems, and methods for cell culture and production of biologics. Disclosed herein are multi-purpose assemblies that facilitate combination of perfusion processes and harvest processes. The multi-purpose assemblies and systems comprising the same simplify and streamline the biomanufacturing process, at least by improving automation capabilities, reducing physical footprint, reducing risk of contamination, and reducing consumable use rates. Also disclosed herein are methods of using the aforementioned assemblies and systems.

### CROSS-REFERENCE

The present application is a non-provisional of 63/357,593, filed Jun. 30, 2022, entitled “SYSTEMS, APPARATUS, AND METHODS FOR CELL CULTURE” and is herein incorporated by reference in its entirety for all purposes.

### FIELD

The present disclosure generally relates to apparatus, systems, and methods for production of biologics. More particularly, the present disclosure relates to improvements in utilization of cell retention devices in biomanufacturing apparatus, systems, and methods.

### BACKGROUND

Cell culture, for example the culture of mammalian, bacterial or fungal cells, may be carried out to harvest the living cells for therapeutic purposes and/or to harvest biological molecules, such as proteins or chemicals produced by the cells (e.g., biologics). During biomanufacturing processes comprising cell culture, filtration is performed to separate, clarify, modify, and/or concentrate a fluid solution, mixture or suspension. In the biotechnology and pharmaceutical industries, filtration is vital for the successful production, processing, and testing of new drugs, diagnostics and other biologics. For example, in the process of manufacturing biologics using animal or microbial cell culture, filtration is done for clarification, selective removal, and/or concentration of certain constituents from the culture media and/or to modify the media prior to further processing. Filtration may also be used to enhance productivity by maintaining a culture in perfusion at high cell concentrations.

One use of filtration units can be as Cell retention devices (CRD). Notable CRDs include tangential flow filtration (also referred to as cross-flow filtration or TFF) systems. Such systems are widely used in the separation of particulates suspended in a liquid phase, and have important biomanufacturing applications. In contrast to dead-end filtration systems in which a single fluid feed is passed through a filter, tangential flow systems are characterized by fluid feeds that flow across a surface of the filter, resulting in the separation of the feed into two components: a permeate component which has passed through the filter, and a retentate component which has not. Compared to dead-end systems, TFF systems are less prone to fouling. Fouling of TFF systems may be reduced further by alternating the direction of the fluid feed across the filtration element as is done in the alternating tangential flow (ATF) technology, by backwashing the permeate through the filter, and/or by periodic washing of the filter.

Advancements in filtration have been made (see e.g., Tim Sewerin et al., “Advances and Applications of Hollow Fiber Nanofiltration Membranes: A Review. Membranes, 2021, 11, 890). One such advancement includes Hollow Fiber (HF) filters (HFF), which may be operated in tangential flow mode, an example of which are tangential flow depth filtration® (TFDF®) filters. Such TFDF® filters combine the benefits of tangential flow with depth filtration. However, certain HF filters can be considered expensive and/or relatively bulky, and during the transition between bioreactors for cell growth phase and harvest phase, cell culture is prone to contamination and associated loss of product.

Thus, it is desirable to have assemblies, systems, and methods capable of improving these processes.

### SUMMARY

Described herein are assemblies, systems, and methods suitable for production of biologics. Disclosed herein are bioreactor systems, comprising: a) a bioreactor including an input port and an output port; b) a feed stream conduit operably connected to the output port and inlet of a Hollow Fiber (HF) filter, the feed stream conduit operable to carry fluid from the bioreactor to the HF filter; c) a retentate stream conduit operably connected to the input port of the bioreactor and an outlet of the HF filter, the retentate stream conduit operable to carry fluid to the bioreactor from the HF filter; and d) a multi-purpose assembly operably connected to the HF filter, the multi-purpose assembly including a first flow path and a second flowpath: wherein the first flowpath includes a first pump operable to draw fluid from the HF filter and a flow meter operable to measure flow rate of fluid in the first flowpath; wherein the second flowpath includes a second pump operable to draw fluid from the HF filter; and wherein the first pump and the second pump are configured to have different pump capacities.

In some embodiments, the HF filter is operable for tangential flow. In some embodiments, the HF filter is a tangential flow depth filtration® (TFDF®) filter. In some embodiments, the first and second pump are configured to have different accuracy ratings. In some embodiments, the system further includes: one or more clarification filter(s) in the first and/or second flow path. In some embodiments, the first and second pumps are selected from the groups consisting of peristaltic, centrifugal, magnetic drive, positive displacement, membrane, pressure-based, Quantex™ (e.g., positive displacement rotary pumps), gear, diaphragm, syringe, and piston pumps. In some embodiments, the first pump and second pumps are peristaltic pumps. In some embodiments, the feed stream conduit comprises a feed stream pump selected from the groups consisting of peristaltic, centrifugal, magnetic drive, positive displacement, membrane, pressure-based, Quantex™, gear, diaphragm, syringe, and piston pumps, is operable to carry unfiltered fluid from the bioreactor to the HF filter. In some embodiments, the retentate stream conduit is operable to carry cell culture fluid to the bioreactor from the HF filter.

In some embodiments, the first flowpath including a first pump operable to draw fluid from the HF filter, draws perfusion permeate stream fluid from the HF filter. In some embodiments, the second flowpath including a second pump operable to draw fluid from the HF filter, draws harvest permeate stream fluid from the HF filter. In some embodiments, the first flowpath and the second flowpath have different internal diameters. In some embodiments, the first flowpath and the second flowpath have the same internal diameters. In some embodiments, the second flowpath is capable of conducting fluids at a higher flowrate than the first flowpath.

In some embodiments, the first pump is operable to draw fluid from the HF filter at a flow rate of between about 0.01 liters per min (LPM) and 5 LPM, between about 0.01 LPM to about 10 LPM, and/or between about 0.01 LPM to about 15 LPM. In some embodiments, the first flowpath and pump are operable to draw a feed flow rate from the HF filter of about 0.8 to about 2.2 liters per fiber per minute (L/fiber/min). In some embodiments, the first pump is operable to draw fluid from the HF filter while maintaining a viable cell density (VCD) of greater than about 75×10⁶ cells/mL, greater than about 50×10⁶ cells/mL, and/or greater than about 25×10⁶ cells/mL. In some embodiments, the first pump is operable to draw fluid from the HF filter at a rate of between about 0.1 and about 5 bioreactor vessel volumes per day (VVD), at a rate of between about 0.5 and about 4.3 bioreactor VVD, at a rate of between about 1 and about 4.3 bioreactor VVD, and/or at a rate up to about 5 bioreactor VVD. In some embodiments, the first pump is operable to draw fluid from the HF filter at a filter flux rate of between about 50 and about 800 liters per square meter per hour (LMH), and/or between about 100 and about 600 LMH.

In some embodiments, the flow meter operable to measure flow rate of fluid in the first flowpath continuously monitors the VVD rate and is in communication with the first pump to adjust the first pump rate to maintain a desired VVD rate. In some embodiments, the flow meter operable to measure flow rate of fluid in the first flowpath is capable of accurately monitoring a flow rate of about 0 to about 8 LPM, or about 0.5 LPM to about 6 LPM. In some embodiments, the first pump is operable to draw fluid from the HF filter at a throughput equal to between about 10,000 liters per square meter (L/m²) to about 30,000 L/m², between about 10,000 liters per square meter (L/m²) to about 50,000 L/m², and/or between about 10,000 liters per square meter (L/m²) to about 70,000 L/m². In some embodiments, the first pump is operable to draw fluid from the HF filter while maintaining a shear rate (s−1) of less than about 5,000 s−1, less than about 3,500 s−1, and/or less than about 2,500 s−1.

In some embodiments, the bioreactor has a volume less than or equal to about 15 liters, less than or equal to about 50 liters, less than or equal to about 100 liters, or less than or equal to about 500 liters. In some embodiments, the bioreactor has a volume greater than or equal to about 500 liters, about 1,000 liters, about 1,500 liters, and/or about 3,500 liters. In some embodiments, the bioreactor has a volume equal to between about 2,000 liters and about 7,000 liters. In some embodiments, the bioreactor has at least two input ports and at least two output ports, and at least two of b), c), and d).

In some embodiments, the bioreactor system is capable of operating in a perfusion process. In some embodiments, the bioreactor system is capable of operating in a harvest process. In some embodiments, the bioreactor system is capable of operating in a perfusion process, an in-between perfusion and harvest process, and a harvest process. In some embodiments, the bioreactor system is capable of operating in a continuous harvest process. In some embodiments, the bioreactor system further comprises a human machine interface (HMI) control unit. In some embodiments, the HMI control unit is programmed and can display perfusion process controls, in-between perfusion and harvest process controls, or harvest process controls.

In some embodiments, the bioreactor system further comprises one or more sensors. In some embodiments, the bioreactor system further comprises one or more of a feed stream or retentate stream fluid pump. In some embodiments, the bioreactor system further comprises at least one component capable of facilitating at least one process intensification parameter. In some embodiments, the process intensification parameter is one or more of increased cell number, increased cell density, provision of rich cell culture growth media, rapid cell number expansion, or increased biologic production. In some embodiments, the bioreactor system is further capable of backflushing the HF filter with permeate fluid.

In some embodiments, the bioreactor system further comprises a second flow meter operable to measure flow rate of fluid in the second flowpath and accurately monitoring a flow rate of about 0 to about 10 LPM, or about 0.5 LPM to about 8 LPM. In some embodiments, the second pump is operable to draw fluid from the HF filter at a throughput equal to between about 12,000 liters per square meter (L/m²) to about 36,000 L/m², between about 12,000 liters per square meter (L/m²) to about 60,000 L/m², and/or between about 10,000 liters per square meter (L/m²) to about 70,000 L/m². In some embodiments, the second pump is operable to draw fluid from the HF filter at a filter flux rate of between about 150 and about 900 liters per square meter per hour (LMH), and/or between about 200 and about 700 LMH.

In some embodiments, the second flowpath and pump are operable to draw a feed flow rate from the HF filter of about 1 to about 3 liters per fiber per minute (L/fiber/min). In some embodiments, the second pump is operable to draw fluid from the HF filter at a flow rate of between about 0.01 LPM to about 8 LPM, between about 0.01 LPM to about 13 LPM, and/or between about 0.01 LPM to about 18 LPM.

In some embodiments, the first flowpath comprises a connection system with ⅜″ ID tubing, and/or ⅛″ ID tubing. In some embodiments, the second flowpath comprises a connection system with ½″ ID tubing and/or ⅛″ ID tubing. In some embodiments, the first flowpath has an accuracy requirement of about 1%. In some embodiments, the second flowpath has an accuracy requirement of about 3%.

In some embodiments, the HF filter is constructed of and/or comprises polypropylene, and/or polyethylene terephthalate. In some embodiments, the HF filter comprises isotropic pore structures. In some embodiments, the HF filter has an average pore size diameter of between about 0.65 μm to about 8 μm. In some embodiments, the HF filter has an average pore size diameter of about 2 μm to about 5 μm. In some embodiments, the HF filter has an average pore size diameter of about 2 μm.

In some embodiments, the bioreactor system is operable for use with shear sensitive cells. In some embodiments, the bioreactor system is operable for use with animal cells. In some embodiments, the bioreactor system is operable for use with mammalian cells. In some embodiments, the bioreactor system is operable for use with Chinese Ovarian Hamster (CHO) cells. In some embodiments, the bioreactor system is operable for use with human embryonic kidney 293 (HEK293) cells. In some embodiments, the bioreactor system is operable for use with cells for the production of a biologic. In some embodiments, the biologic comprises an antibody, peptide, and/or virus.

Also disclosed herein are multi-purpose filter assemblies, comprising: a) a Hollow Fiber (HF) filter; and b) a multi-purpose assembly operably connected to the HF filter, the multi-purpose assembly including a first flow path and a second flowpath: wherein the first flowpath includes a first pump operable to draw fluids from the HF filter and a flow meter operable to measure flow rate of fluid in the first flowpath; wherein the second flowpath includes a second pump operable to draw fluid from the HF filter; and wherein the first pump and the second pump are capable of having different pump capacities and accuracy ratings. In some embodiments, the HF filter is operable for tangential flow.

In some embodiments, the HF filter is a tangential flow depth filtration® (TFDF®) filter. In some embodiments, the multi-purpose filter assembly further comprises one or more clarification filter(s) in the first and/or second flow path. In some embodiments, the first and second pumps are selected from the groups consisting of peristaltic, centrifugal, magnetic drive, positive displacement, membrane, pressure-based, Quantex™, gear, diaphragm, syringe, and piston pumps. In some embodiments, the first pump and second pumps are peristaltic pumps. In some embodiments, the first flowpath and pump are operable to draw fluids from the HF filter at a throughput greater than or equal to about 10,000 liters per square meter (L/m²) to about 30,000 L/m². In some embodiments, the first flowpath and pump are operable to draw fluids from the HF filter at a throughput greater than or equal to about 10,000 liters per square meter (L/m²) to about 50,000 L/m². In some embodiments, the first flowpath and pump are operable to draw fluids from the HF filter at a throughput greater than or equal to about 10,000 liters per square meter (L/m²) to about 70,000 L/m². In some embodiments, the first flowpath and pump are operable to draw a feed flow rate from the HF filter of about 0.8 to about 2.2 liters per fiber per minute (L/fiber/min). In some embodiments, the first flowpath and pump are operable to draw a feed flow rate from the HF filter of about 0.8 to about 2.0 L/fiber/min, and/or about 1 to about 1.8 L/fiber/min. In some embodiments, the first flowpath and pump are operable to draw fluids from the HF filter at a filter flux of about 50 to about 800 LMH, and/or about 100 to about 600 LMH. In some embodiments, the first flowpath and pump are operable to maintain a packed cell volume (PCV) of about 2 to about 40%, about 8 to about 40%, and/or about 12 to about 35%. In some embodiments, the first flowpath and pump are operable to draw fluid from the HF filter while maintaining a VCD of greater than about 25×10⁶ cells/mL, about 50×10⁶ cells/mL, about 75×10⁶ cells/mL, about 100×10⁶ cells/mL, and/or about 25×10⁷ cells/mL. In some embodiments, the first flowpath and pump are operable to draw fluid from the HF filter at a shear rate of less than about 5,000 s−1, about 3,500 s−1, and/or about 2,500 s−1. In some embodiments, the first flowpath and pump are operable to draw fluid from the HF filter at a flow rate of between about 0.01 LPM to about 5 LPM, about 0.01 LPM to about 10 LPM, and/or about 0.01 LPM to about 15 LPM.

In some embodiments, the flow meter operable to measure flow rate of fluid in the first flowpath continuously monitors the LPM rate and is in communication with the first pump to adjust the first pump rate to maintain a desired LPM rate. In some embodiments, the flow meter operable to measure flow rate of fluid in the first flowpath is capable of accurately monitoring a flow rate of about 0 to about 8 LPM, and/or about 0.01 LPM to about 6 LPM. In some embodiments, the first flowpath has an accuracy requirement of about 1%. In some embodiments, the second flowpath has an accuracy requirement of about 3%.

In some embodiments, the first flowpath and the second flowpath have different internal diameters. In some embodiments, the first flowpath and the second flowpath have the same internal diameters. In some embodiments, the second flowpath and pump are operable to draw fluid from the HF filter at a higher flowrate than the first flowpath and first pump. In some embodiments, the second flowpath and pump are operable to draw fluid from the HF filter as a continuous harvest process. In some embodiments, the HF filter is not replaced when the multi-purpose assembly directs fluids through one flowpath and pump and then directs fluid through the other flowpath and pump. In some embodiments, the HF filter is not replaced when the multi-purpose assembly directs fluids first through the first flowpath and pump, and subsequently through the second flowpath and pump. In some embodiments, the first flowpath and second flowpath of the multi-purpose assembly are connected by a T connector, a Y connector, or a valve.

In some embodiments, the multi-purpose filter assembly further comprises a second flow meter operable to measure flow rate of fluid in the second flowpath and accurately monitoring a flow rate of about 0 to about 10 LPM, or about 0.5 LPM to about 8 LPM. In some embodiments, the second pump is operable to draw fluid from the HF filter at a throughput equal to between about 12,000 liters per square meter (L/m²) to about 36,000 L/m², between about 12,000 L/m² to about 60,000 L/m², and/or between about 10,000 liters per square meter (L/m²) to about 70,000 L/m². In some embodiments, the second pump is operable to draw fluid from the HF filter at a filter flux rate of between about 150 and about 900 liters per square meter per hour (LMH), and/or between about 200 and about 700 LMH. In some embodiments, the second flowpath and pump are operable to draw a feed flow rate from the HF filter of about 1 to about 3 liters per fiber per minute (L/fiber/min). In some embodiments, the second pump is operable to draw fluid from the HF filter at a flow rate of between about 0.01 LPM to about 8 LPM, about 0.01 LPM to about 13 LPM, and/or about 0.01 LPM to about 18 LPM.

In some embodiments, the HF filter is constructed of and/or comprises polypropylene, and/or polyethylene terephthalate. In some embodiments, the HF filter comprises isotropic pore structures. In some embodiments, the HF filter has an average pore size diameter of between about 0.65 μm to about 8 μm. In some embodiments, the HF filter has an average pore size diameter of about 2 μm to about 5 μm. In some embodiments, the HF filter has an average pore size diameter of about 2 μm.

In some embodiments, the multi-purpose filter assembly is operable for use with shear sensitive cells, animal cells, mammalian cells, CHO cells, and/or HEK293 cells. In some embodiments, the multi-purpose filter assembly is operable for use with cells for the production of a biologic. In some embodiments, the biologic comprises an antibody, peptide, and/or virus.

Also disclosed herein are methods of producing biologics from cells, comprising: a) expanding cells in a cell culture fluid in a bioreactor, b) during cell expansion, perfusing cell culture fluid through a Hollow Fiber (HF) filter to remove spent cell culture media while retaining the expanding cells, and adding an appropriate replacement volume of cell culture media to the bioreactor to maintain a desired cell culture fluid level in the bioreactor, wherein the spent cell culture media is perfused through the HF filter at a first flow rate to enter a first flowpath, and c) following cell expansion, harvesting cell culture fluid through a HF filter to obtain the biologics, and adding an appropriate replacement volume of cell culture media to the bioreactor to maintain a desired cell culture fluid level in the bioreactor during different phases of harvest, wherein the cell culture media is harvested through the HF filter at a second flow rate to enter a second flowpath, wherein the second flow rate is greater than the first flow rate. In some embodiments, the HF filter is operable for tangential flow. In some embodiments, the HF filter is a tangential flow depth filtration® (TFDF®) filter. In some embodiments, the replacement volume of cell culture media in b) and/or c) is the same amount of spent cell culture media as is removed. In some embodiments, the replacement volume of cell culture media in b) and/or c) is greater than the amount of spent cell culture media removed. In some embodiments, the replacement volume of cell culture media in b) and/or c) is less than the amount of spent cell culture media removed. In some embodiments, the replacement volume of cell culture media is fresh cell culture media. In some embodiments, the first flow rate is less than or equal to about 5 LPM, and a second flow rate is greater than about 5 LPM. In some embodiments, the HF filter is constructed of and/or comprises polypropylene, and/or polyethylene terephthalate. In some embodiments, the HF filter comprises isotropic pore structures. In some embodiments, the HF filter has an average pore size diameter of between about 0.65 μm to about 8 μm. In some embodiments, the HF filter has an average pore size diameter of about 2 μm to about 5 μm. In some embodiments, the HF filter has an average pore size diameter of about 2 μm. In some embodiments, the cells are shear sensitive cells. In some embodiments, the cells are animal cells, mammalian cells, CHO cells, and/or HEK293 cells. In some embodiments, the biologic comprises an antibody, peptide, and/or virus.

Also disclosed herein are methods of producing biologics from cells, comprising use of a bioreactor system and/or multi-purpose filter assembly disclosed herein.

Also disclosed herein are uses of a multi-purpose filter assembly disclosed herein in a bioreactor system. In some embodiments, the bioreactor system is a bioreactor system of the disclosure or invention.

Also disclosed herein are uses of a bioreactor system disclosed herein for the culturing of cells. In some embodiments, the cells produce biologics and the use may further comprise harvesting cell culture fluid through the HF filter to obtain the biologics.

Also disclosed herein are methods of producing biologics from cells, comprising use of a bioreactor system or multi-purpose filter assembly disclosed herein. Also disclosed herein are methods of use of a system, assembly, or method disclosed herein to improve productivity, improve performance, improve efficiency, reduce contamination rates, reduce capital investment, reduce physical footprint, reduce consumables attrition, and/or reduce operation costs for a facility comprising a biologics production process.

Certain embodiments of the present invention are characterized through the following aspects.

Aspect 1 is a bioreactor system, comprising: a) a bioreactor including an input port and an output port; b) a feed stream conduit operably connected to the output port and inlet of a Hollow Fiber (HF) filter, the feed stream conduit operable to carry fluid from the bioreactor to the HF filter; c) a retentate stream conduit operably connected to the input port of the bioreactor and an outlet of the HF filter, the retentate stream conduit operable to carry fluid to the bioreactor from the HF filter; and d) a multi-purpose assembly operably connected to the HF filter, the multi-purpose assembly including a first flow path and a second flowpath: wherein the first flowpath includes a first pump operable to draw fluid from the HF filter and a flow meter operable to measure flow rate of fluid in the first flowpath; wherein the second flowpath includes a second pump operable to draw fluid from the HF filter; and wherein the first pump and the second pump are configured to have different pump capacities.

Aspect 2 is the bioreactor system of aspect 1, wherein the HF filter is operable for tangential flow.

Aspect 3 is the bioreactor system of aspects 1 or 2, wherein the HF filter is a tangential flow depth filtration® (TFDF®) filter.

Aspect 4 is the bioreactor system of any one of aspects 1-3, wherein the first and second pump are configured to have different accuracy ratings.

Aspect 5 is the bioreactor system of any one of aspects 1-4, further including: one or more clarification filter(s) in the first and/or second flow path.

Aspect 6 is the bioreactor system of any one of aspects 1-5, wherein the first and second pumps are selected from the groups consisting of peristaltic, centrifugal, magnetic drive, positive displacement, membrane, pressure-based, Quantex™, gear, diaphragm, syringe, and piston pumps.

Aspect 7 is the bioreactor system of any one of aspects 1-6, wherein the first pump and second pumps are peristaltic pumps.

Aspect 8 is the bioreactor system of any one of aspects 1-7, wherein the feed stream conduit comprises a feed stream pump selected from the groups consisting of peristaltic, centrifugal, magnetic drive, positive displacement, membrane, pressure-based, Quantex™ gear, diaphragm, syringe, and piston pumps, is operable to carry unfiltered fluid from the bioreactor to the HF filter.

Aspect 9 is the bioreactor system of any one of aspects 1-8, wherein the retentate stream conduit is operable to carry cell culture fluid to the bioreactor from the HF filter.

Aspect 10 is the bioreactor system of any one of aspects 1-9, wherein the first flowpath including a first pump operable to draw fluid from the HF filter, draws perfusion permeate stream fluid from the HF filter.

Aspect 11 is the bioreactor system of any one of aspects 1-10, wherein the second flowpath including a second pump operable to draw fluid from the HF filter, draws harvest permeate stream fluid from the HF filter.

Aspect 12 is the bioreactor system of any one of aspects 1-11, wherein the first flowpath and the second flowpath have the same or have different internal diameters.

Aspect 13 is the bioreactor system of any one of aspects 1-12, wherein the second flowpath is capable of conducting fluids at a higher flowrate than the first flowpath.

Aspect 14 is the bioreactor system of any one of aspects 1-13, wherein the first pump is operable to draw fluid from the HF filter at a flow rate of between about 0.01 liters per min (LPM) and 5 LPM.

Aspect 15 is the bioreactor system of any one of aspects 1-13, wherein the first pump is operable to draw fluid from the HF filter at a flow rate of between about 0.01 LPM to about 10 LPM.

Aspect 16 is the bioreactor system of any one of aspects 1-13, wherein the first pump is operable to draw fluid from the HF filter at a flow rate of between about 0.01 LPM to about 15 LPM.

Aspect 17 is the bioreactor system of any one of aspects 1-16, wherein the first flowpath and pump are operable to draw a feed flow rate from the HF filter of about 0.8 to about 2.2 liters per fiber per minute (L/fiber/min).

Aspect 18 is the bioreactor system of any one of aspects 1-17, wherein the first pump is operable to draw fluid from the HF filter while maintaining a viable cell density (VCD) of greater than about 75×10⁶ cells/mL.

Aspect 19 is the bioreactor system of any one of aspects 1-17, wherein the first pump is operable to draw fluid from the HF filter while maintaining a viable cell density (VCD) of greater than about 50×10⁶ cells/mL.

Aspect 20 is the bioreactor system of any one of aspects 1-17, wherein the first pump is operable to draw fluid from the HF filter while maintaining a VCD of greater than about 25×10⁶ cells/mL.

Aspect 21 is the bioreactor system of any one of aspects 1-20, wherein the first pump is operable to draw fluid from the HF filter at a rate of between about 0.1 and about 5 bioreactor vessel volumes per day (VVD).

Aspect 22 is the bioreactor system of any one of aspects 1-21, wherein the first pump is operable to draw fluid from the HF filter at a rate of between about 0.5 and about 4.3 bioreactor vessel volumes per day (VVD).

Aspect 23 is the bioreactor system of any one of aspects 1-22, wherein the first pump is operable to draw fluid from the HF filter at a filter flux rate of between about 50 and about 800 liters per square meter per hour (LMH).

Aspect 24 is the bioreactor system of any one of aspects 1-23, wherein the first pump is operable to draw fluid from the HF filter at a filter flux rate of between about 100 and about 600 LMH.

Aspect 25 is the bioreactor system of any one of aspects 1-24, wherein the flow meter operable to measure flow rate of fluid in the first flowpath continuously monitors the VVD rate and is in communication with the first pump to adjust the first pump rate to maintain a desired VVD rate.

Aspect 26 is the bioreactor system of any one of aspects 1-25, wherein the flow meter operable to measure flow rate of fluid in the first flowpath is capable of accurately monitoring a flow rate of about 0 to about 8 LPM, or about 0.5 LPM to about 6 LPM.

Aspect 27 is the bioreactor system of any one of aspects 1-26, wherein the first pump is operable to draw fluid from the HF filter at a throughput equal to between about 10,000 liters per square meter (L/m²) to about 30,000 L/m².

Aspect 28 is the bioreactor system of any one of aspects 1-26, wherein the first pump is operable to draw fluid from the HF filter at a throughput equal to between about 10,000 liters per square meter (L/m²) to about 70,000 L/m².

Aspect 29 is the bioreactor system of any one of aspects 1-28, wherein the first pump is operable to draw fluid from the HF filter while maintaining a shear rate (s−1) of less than about 5,000 s−1.

Aspect 30 is the bioreactor system of any one of aspects 1-29, wherein the first pump is operable to draw fluid from the HF filter while maintaining a shear rate (s−1) of less than about 3,500 s−1.

Aspect 31 is the bioreactor system of any one of aspects 1-30, wherein the first pump is operable to draw fluid from the HF filter while maintaining a shear rate (s−1) of less than about 2,500 s−1.

Aspect 32 is the bioreactor system of any one of aspects 1-31, wherein the bioreactor has a volume less than or equal to about 15 liters, less than or equal to about 50 liters, less than or equal to about 100 liters, or less than or equal to about 500 liters.

Aspect 33 is the bioreactor system of any one of aspects 1-31, wherein the bioreactor has a volume greater than or equal to about 500 liters.

Aspect 34 is the bioreactor system of any one of aspects 1-31, wherein the bioreactor has a volume greater than or equal to about 1,000 liters.

Aspect 35 is the bioreactor system of any one of aspects 1-31, wherein the bioreactor has a volume greater than or equal to about 1,500 liters.

Aspect 36 is the bioreactor system of any one of aspects 1-31, wherein the bioreactor has a volume equal to between about 2,000 liters and about 3,500 liters.

Aspect 37 is the bioreactor system of any one of aspects 1-31, wherein the bioreactor has a volume equal to between about 2,000 liters and about 7,000 liters.

Aspect 38 is the bioreactor system of any one of aspects 1-37, wherein the bioreactor has at least two input ports and at least two output ports, and at least two of b), c), and d).

Aspect 39 is the bioreactor system of any one of aspects 1-38, wherein the bioreactor system is capable of operating in a perfusion process.

Aspect 40 is the bioreactor system of any one of aspects 1-39, wherein the bioreactor system is capable of operating in a harvest process.

Aspect 41 is the bioreactor system of any one of aspects 1-40, wherein the bioreactor system is capable of operating in a perfusion process, an in-between perfusion and harvest process, and a harvest process.

Aspect 41.1 is the bioreactor system of any one of aspects 1-41, wherein the bioreactor system is capable of operating in more than one perfusion process.

Aspect 41.2 is the bioreactor system of any one of aspects 1-41.1, wherein the bioreactor system is capable of operating in more than one perfusion process, an in-between perfusion and harvest process, and a harvest process.

Aspect 42 is the bioreactor system of any one of aspects 1-41.2, wherein the bioreactor system is capable of operating in a continuous harvest process.

Aspect 43 is the bioreactor system of any one of aspects 1-42, further comprising a human machine interface (HMI) control unit.

Aspect 44 is the bioreactor system of aspect 43, wherein the HMI control unit is programmed and can display perfusion process controls, in-between perfusion and harvest process controls, or harvest process controls.

Aspect 45 is the bioreactor system of any one of aspects 1-44, further comprising one or more sensors.

Aspect 46 is the bioreactor system of any one of aspects 1-46, further comprising one or more of a feed stream or retentate stream fluid pump.

Aspect 47 is the bioreactor system of any one of aspects 1-46, further comprising at least one component capable of facilitating at least one process intensification parameter.

Aspect 48 is the bioreactor system of aspect 47, wherein the process intensification parameter is one or more of increased cell number, increased cell density, provision of rich cell culture growth media, rapid cell number expansion, or increased biologic production.

Aspect 49 is the bioreactor system of any one of aspects 1-48, further capable of backflushing the HF filter with permeate fluid.

Aspect 50 is the bioreactor system of any one of aspects 1-49, further comprising a second flow meter operable to measure flow rate of fluid in the second flowpath and accurately monitoring a flow rate of about 0 to about 10 LPM, or about 0.5 LPM to about 8 LPM.

Aspect 51 is the bioreactor system of any one of aspects 1-50, wherein the second pump is operable to draw fluid from the HF filter at a throughput equal to between about 12,000 liters per square meter (L/m²) to about 36,000 L/m².

Aspect 52 is the bioreactor system of any one of aspects 1-50, wherein the second pump is operable to draw fluid from the HF filter at a throughput equal to between about 12,000 liters per square meter (L/m²) to about 60,000 L/m², or equal to between about 12,000 liters per square meter (L/m²) to about 70,000 L/m².

Aspect 53 is the bioreactor system of any one of aspects 1-52, wherein the second pump is operable to draw fluid from the HF filter at a filter flux rate of between about 150 and about 900 liters per square meter per hour (LMH).

Aspect 54 is the bioreactor system of any one of aspects 1-53, wherein the second pump is operable to draw fluid from the HF filter at a filter flux rate of between about 200 and about 700 LMH.

Aspect 55 is the bioreactor system of any one of aspects 1-54, wherein the second flowpath and pump are operable to draw a feed flow rate from the HF filter of about 1 to about 3 liters per fiber per minute (L/fiber/min).

Aspect 56 is the bioreactor system of any one of aspects 1-55, wherein the second pump is operable to draw fluid from the HF filter at a flow rate of between about 0.01 LPM to about 8 LPM.

Aspect 57 is the bioreactor system of any one of aspects 1-55, wherein the second pump is operable to draw fluid from the HF filter at a flow rate of between about 0.01 LPM to about 13 LPM.

Aspect 58 is the bioreactor system of any one of aspects 1-55, wherein the second pump is operable to draw fluid from the HF filter at a flow rate of between about 0.01 LPM to about 18 LPM.

Aspect 59 is the bioreactor system of any one of aspects 1-58, wherein the first flowpath comprises a connection system with ⅜″ ID tubing.

Aspect 60 is the bioreactor system of any one of aspects 1-58, wherein the first flowpath comprises a connection system with ⅛″ ID tubing.

Aspect 61 is the bioreactor system of any one of aspects 1-60, wherein the second flowpath comprises a connection system with ½″ ID tubing.

Aspect 62 is the bioreactor system of any one of aspects 1-60, wherein the second flowpath comprises a connection system with ⅛″ ID tubing.

Aspect 63 is the bioreactor system of any one of aspects 1-62, wherein the first flowpath has an accuracy requirement of about 1%.

Aspect 64 is the bioreactor system of any one of aspects 1-63, wherein the second flowpath has an accuracy requirement of about 3%.

Aspect 65 is the bioreactor system of any one of aspects 1-64, wherein the HF filter is constructed of and/or comprises polypropylene, and/or polyethylene terephthalate.

Aspect 66 is the bioreactor system of any one of aspects 1-65, wherein the HF filter comprises isotropic pore structures, and/or comprises an average pore lumen diameter of about 0.65 μm to about 8 μm, or about 2 μm to about 5 μm.

Aspect 67 is the bioreactor system of any one of aspects 1-66, operable for use with shear sensitive cells.

Aspect 68 is the bioreactor system of any one of aspects 1-67, operable for use with animal cells.

Aspect 69 is the bioreactor system of any one of aspects 1-68, operable for use with mammalian cells.

Aspect 70 is the bioreactor system of any one of aspects 1-69, operable for use with Chinese Ovarian Hamster (CHO) cells.

Aspect 71 is the bioreactor system of any one of aspects 1-69, operable for use with human embryonic kidney 293 (HEK293) cells.

Aspect 72 is the bioreactor system of any one of aspects 1-71, operable for use with cells for the production of a biologic.

Aspect 73 is the bioreactor system of aspect 72, wherein the biologic comprises an antibody, peptide, and/or virus.

Aspect 74 is a multi-purpose filter assembly, comprising: a) a Hollow Fiber (HF) filter; and b) a multi-purpose assembly operably connected to the HF filter, the multi-purpose assembly including a first flow path and a second flowpath: wherein the first flowpath includes a first pump operable to draw fluids from the HF filter and a flow meter operable to measure flow rate of fluid in the first flowpath; wherein the second flowpath includes a second pump operable to draw fluid from the HF filter; and wherein the first pump and the second pump are capable of having different pump capacities and accuracy ratings.

Aspect 75 is the multi-purpose filter assembly of aspect 74, wherein the HF filter is operable for tangential flow.

Aspect 76 is the multi-purpose filter assembly of aspects 74 or 75, wherein the HF filter is a tangential flow depth filtration® (TFDF®) filter.

Aspect 77 is the multi-purpose filter assembly of any one of aspects 74-76, further including: one or more clarification filter(s) in the first and/or second flow path.

Aspect 78 is the multi-purpose filter assembly of any one of aspects 74-77, wherein the first and second pumps are selected from the groups consisting of peristaltic, centrifugal, magnetic drive, positive displacement, membrane, pressure-based, Quantex™, gear, diaphragm, syringe, and piston pumps.

Aspect 79 is the multi-purpose filter assembly of any one of aspects 74-78, wherein the first pump and second pumps are peristaltic pumps.

Aspect 80 is the multi-purpose filter assembly of any one of aspects 74-79, wherein the first flowpath and pump are operable to draw fluids from the HF filter at a throughput greater than or equal to about 10,000 liters per square meter (L/m²) to about 30,000 L/m².

Aspect 81 is the multi-purpose filter assembly of any one of aspects 74-79, wherein the first flowpath and pump are operable to draw fluids from the HF filter at a throughput greater than or equal to about 10,000 liters per square meter (L/m²) to about 70,000 L/m².

Aspect 82 is the multi-purpose filter assembly of any one of aspects 74-81, wherein the first flowpath and pump are operable to draw a feed flow rate from the HF filter of about to about 2.2 liters per fiber per minute (L/fiber/min).

Aspect 83 is the multi-purpose filter assembly of any one of aspects 74-82, wherein the first flowpath and pump are operable to draw a feed flow rate from the HF filter of about to about 2.0 L/fiber/min.

Aspect 84 is the multi-purpose filter assembly of any one of aspects 74-83, wherein the first flowpath and pump are operable to draw a feed flow rate from the HF filter of about 1 to about 1.8 L/fiber/min.

Aspect 85 is the multi-purpose filter assembly of any one of aspects 74-84, wherein the first flowpath and pump are operable to draw fluids from the HF filter at a filter flux of about 50 to about 800 LMH.

Aspect 86 is the multi-purpose filter assembly of any one of aspects 74-85, wherein the first flowpath and pump are operable to draw fluids from the HF filter at a filter flux of about 100 to about 600 LMH.

Aspect 87 is the multi-purpose filter assembly of any one of aspects 74-86, wherein the first flowpath and pump are operable to maintain a packed cell volume (PCV) of about 2 to about 40%.

Aspect 88 is the multi-purpose filter assembly of any one of aspects 74-87, wherein the first flowpath and pump are operable to maintain a PCV of about 8 to about 40%.

Aspect 89 is the multi-purpose filter assembly of any one of aspects 74-88, wherein the first flowpath and pump are operable to maintain a PCV of about 12 to about 35%.

Aspect 90 is the multi-purpose filter assembly of any one of aspects 74-89, wherein the first flowpath and pump are operable to draw fluid from the HF filter while maintaining a VCD of greater than about 25×10⁶ cells/mL.

Aspect 91 is the multi-purpose filter assembly of any one of aspects 74-90, wherein the first flowpath and pump are operable to draw fluid from the HF filter while maintaining a VCD of greater than about 50×10⁶ cells/mL.

Aspect 92 is the multi-purpose filter assembly of any one of aspects 74-91, wherein the first flowpath and pump are operable to draw fluid from the HF filter while maintaining a VCD of greater than about 75×10⁶ cells/mL.

Aspect 93 is the multi-purpose filter assembly of any one of aspects 74-92, wherein the first flowpath and pump are operable to draw fluid from the HF filter while maintaining a VCD of greater than about 100×10⁶ cells/mL.

Aspect 94 is the multi-purpose filter assembly of any one of aspects 74-93, wherein the first flowpath and pump are operable to draw fluid from the HF filter while maintaining a VCD of greater than about 25×10⁷ cells/mL.

Aspect 95 is the multi-purpose filter assembly of any one of aspects 74-94, wherein the first flowpath and pump are operable to draw fluid from the HF filter at a shear rate of less than about 5,000 s−1.

Aspect 96 is the multi-purpose filter assembly of any one of aspects 74-95, wherein the first flowpath and pump are operable to draw fluid from the HF filter at a shear rate of less than about 3,500 s−1.

Aspect 97 is the multi-purpose filter assembly of any one of aspects 74-96, wherein the first flowpath and pump are operable to draw fluid from the HF filter at a shear rate of less than about 2,500 s−1.

Aspect 98 is the multi-purpose filter assembly of any one of aspects 74-97, wherein the first flowpath and pump are operable to draw fluid from the HF filter at a flow rate of between about 0.01 LPM to about 5 LPM.

Aspect 99 is the multi-purpose filter assembly of any one of aspects 74-97, wherein the first flowpath and pump are operable to draw fluid from the HF filter at a flow rate of between about 0.01 LPM to about 10 LPM.

Aspect 100 is the multi-purpose filter assembly of any one of aspects 74-97, wherein the first flowpath and pump are operable to draw fluid from the HF filter at a flow rate of between about 0.01 LPM to about 15 LPM.

Aspect 101 is the multi-purpose filter assembly of any one of aspects 74-100, wherein the flow meter operable to measure flow rate of fluid in the first flowpath continuously monitors the LPM rate and is in communication with the first pump to adjust the first pump rate to maintain a desired LPM rate.

Aspect 102 is the multi-purpose filter assembly of any one of aspects 74-101, wherein the flow meter operable to measure flow rate of fluid in the first flowpath is capable of accurately monitoring a flow rate of about 0 to about 8 LPM.

Aspect 103 is the multi-purpose filter assembly of any one of aspects 74-102, wherein the flow meter operable to measure flow rate of fluid in the first flowpath is capable of accurately monitoring a flow rate of about 0.01 LPM to about 6 LPM.

Aspect 104 is the multi-purpose filter assembly of any one of aspects 74-103, wherein the first flowpath has an accuracy requirement of about 1%.

Aspect 105 is the multi-purpose filter assembly of any one of aspects 74-104, wherein the second flowpath has an accuracy requirement of about 3%.

Aspect 106 is the multi-purpose filter assembly of any one of aspects 74-105, wherein the first flowpath and the second flowpath have the same or have different internal diameters.

Aspect 107 is the multi-purpose filter assembly of any one of aspects 74-106, wherein the second flowpath and pump are operable to draw fluid from the HF filter at a higher flowrate than the first flowpath and first pump.

Aspect 108 is the multi-purpose filter assembly of any one of aspects 74-107, wherein the second flowpath and pump are operable to draw fluid from the HF filter as a continuous harvest process.

Aspect 109 is the multi-purpose filter assembly of any one of aspects 74-108, wherein the HF filter is not replaced when the multi-purpose assembly directs fluids through one flowpath and pump and then directs fluid through the other flowpath and pump.

Aspect 110 is the multi-purpose filter assembly of aspect 109, wherein the HF filter is not replaced when the multi-purpose assembly directs fluids first through the first flowpath and pump, and subsequently through the second flowpath and pump.

Aspect 111 is the multi-purpose filter assembly of any one of aspects 74-110, wherein the first flowpath and second flowpath of the multi-purpose assembly are connected by a T connector, a Y connector, or a valve.

Aspect 112 is the multi-purpose filter assembly of any one of aspects 74-111, further comprising a second flow meter operable to measure flow rate of fluid in the second flowpath and accurately monitoring a flow rate of about 0 to about 10 LPM, or about 0.5 LPM to about 8 LPM.

Aspect 113 is the multi-purpose filter assembly of any one of aspects 74-112, wherein the second pump is operable to draw fluid from the HF filter at a throughput equal to between about 12,000 liters per square meter (L/m²) to about 36,000 L/m².

Aspect 114 is the multi-purpose filter assembly of any one of aspects 74-112, wherein the second pump is operable to draw fluid from the HF filter at a throughput equal to between about 12,000 liters per square meter (L/m²) to about 60,000 L/m², and/or between about 10,000 liters per square meter (L/m²) to about 70,000 L/m².

Aspect 115 is the multi-purpose filter assembly of any one of aspects 74-114, wherein the second pump is operable to draw fluid from the HF filter at a filter flux rate of between about 150 and about 900 liters per square meter per hour (LMH).

Aspect 116 is the multi-purpose filter assembly of any one of aspects 74-115, wherein the second pump is operable to draw fluid from the HF filter at a filter flux rate of between about 200 and about 700 LMH.

Aspect 117 is the multi-purpose filter assembly of any one of aspects 74-116, wherein the second flowpath and pump are operable to draw a feed flow rate from the HF filter of about 1 to about 3 liters per fiber per minute (L/fiber/min).

Aspect 118 is the multi-purpose filter assembly of any one of aspects 74-117, wherein the second pump is operable to draw fluid from the HF filter at a flow rate of between about 0.01 LPM to about 8 LPM.

Aspect 119 is the multi-purpose filter assembly of any one of aspects 74-117, wherein the second pump is operable to draw fluid from the HF filter at a flow rate of between about 0.01 LPM to about 13 LPM.

Aspect 120 is the multi-purpose filter assembly of any one of aspects 74-117, wherein the second pump is operable to draw fluid from the HF filter at a flow rate of between about 0.01 LPM to about 18 LPM.

Aspect 121 is the multi-purpose filter assembly of any one of aspects 74-120, wherein the HF filter is constructed of and/or comprises polypropylene, and/or polyethylene terephthalate.

Aspect 122 is the multi-purpose filter assembly of any one of aspects 74-121, wherein the HF filter comprises isotropic pore structures, and/or comprises an average pore lumen diameter of about 0.65 μm to about 8 μm, or about 2 μm to about 5 μm.

Aspect 123 is the multi-purpose filter assembly of any one of aspects 74-122, operable for use with shear sensitive cells.

Aspect 124 is the multi-purpose filter assembly of any one of aspects 74-123, operable for use with animal cells.

Aspect 125 is the multi-purpose filter assembly of any one of aspects 74-124, operable for use with mammalian cells.

Aspect 126 is the multi-purpose filter assembly of any one of aspects 74-125, operable for use with Chinese Ovarian Hamster (CHO) cells.

Aspect 127 is the multi-purpose filter assembly of any one of aspects 74-125, operable for use with human embryonic kidney 293 (HEK293) cells.

Aspect 128 is the multi-purpose filter assembly of any one of aspects 74-127, operable for use with cells for the production of a biologic.

Aspect 129 is the multi-purpose filter assembly of aspect 128, wherein the biologic comprises an antibody, peptide, and/or virus.

Aspect 130 is a method of producing biologics from cells, comprising: a) expanding cells in a cell culture fluid in a bioreactor, b) during cell expansion, perfusing cell culture fluid through a Hollow Fiber (HF) filter to remove spent cell culture media while retaining the expanding cells, and adding an appropriate replacement volume of cell culture media to the bioreactor to maintain a desired cell culture fluid level in the bioreactor, wherein the spent cell culture media is perfused through the HF filter at a first flow rate to enter a first flowpath, and c) following cell expansion, harvesting cell culture fluid through a HF filter to obtain the biologics, and adding an appropriate replacement volume of cell culture media to the bioreactor to maintain a desired cell culture fluid level in the bioreactor during different phases of harvest, wherein the cell culture media is harvested through the HF filter at a second flow rate to enter a second flowpath, wherein the second flow rate is greater than the first flow rate.

Aspect 131 is the method of aspect 130, wherein the HF filter is operable for tangential flow.

Aspect 132 is the method of any one of aspects 130-131, wherein the HF filter is a tangential flow depth filtration® (TFDF®) filter.

Aspect 133 is the method of any one of aspects 130-132, wherein replacement volume of cell culture media in b) and/or c) is about the same amount of spent cell culture media as is removed.

Aspect 134 is the method of any one of aspects 130-132, wherein the replacement volume of cell culture media in b) and/or c) is greater than the amount of spent cell culture media removed.

Aspect 135 is the method of any one of aspects 130-132, wherein the replacement volume of cell culture media in b) and/or c) is less than the amount of spent cell culture media removed.

Aspect 136 is the method of any one of aspects 130-135, wherein the replacement volume of cell culture media is fresh cell culture media.

Aspect 136.1 is the method of any one of aspects 130-136, wherein the perfusion process comprises more than one perfusion stage.

Aspect 136.2 is the method of any one of aspects 130-136.1, wherein the perfusion process comprises more than one perfusion stage, and the HF filter is used for more than one perfusion stage, an in-between perfusion and harvest process, and a harvest process.

Aspect 137 is the method of any one of aspects 130-136.2, wherein the first flow rate is less than or equal to about 5 LPM, and a second flow rate is greater than about 5 LPM.

Aspect 138 is the method of any one of aspects 130-137, wherein the HF filter is constructed of and/or comprises polypropylene, and/or polyethylene terephthalate.

Aspect 139 is the method of any one of aspects 130-138, wherein the HF filter comprises isotropic pore structures, and/or comprises an average pore lumen diameter of about 0.65 μm to about 8 μm, or about 2 μm to about 5 μm.

Aspect 140 is the method of any one of aspects 130-139, wherein the cells are shear sensitive cells.

Aspect 141 is the method of any one of aspects 130-140, wherein the cells are animal cells.

Aspect 142 is the method of any one of aspects 130-141, wherein the cells are mammalian cells.

Aspect 143 is the method of any one of aspects 130-142, wherein the cells are CHO cells.

Aspect 144 is the method of any one of aspects 130-142, wherein the cells are HEK293 cells.

Aspect 145 is the method of any one of aspects 130-144, wherein the biologic comprises an antibody, peptide, and/or virus.

Aspect 146 is a method of producing biologics from cells, comprising use of a bioreactor system or multi-purpose filter assembly according to any one of aspects 1-129.

Aspect 147 is a use of a multi-purpose filter assembly of any one of aspects 74-129 in a bioreactor system.

Aspect 148 is the use of aspect 147, wherein the bioreactor system comprises the features of any of aspects 1 to 73.

Aspect 149 is a use of a bioreactor system of any one of aspects 1 to 74 for the culturing of cells.

Aspect 150 is the use of aspect 149, wherein the cells produce biologics and the use further comprises harvesting cell culture fluid through the HF filter to obtain the biologics.

Aspect 151 is the system, assembly, method, or use of any one of aspects 1-150, wherein the system, assembly, method, or use results in one or more of improved productivity, improved performance, improved efficiency, reduced contamination rates, reduced capital investment, reduced physical footprint, reduced consumables attrition, or reduced operation costs for a facility comprising a biologics production process.

### BRIEF DESCRIPTION OF THE DRAWINGS

The present disclosure is described in conjunction with the appended figures:

FIG. 1 is a schematic diagram of an exemplary multi-purpose assembly apparatus.

FIG. 2 is a schematic diagram of an exemplary system comprising a multi-purpose assembly apparatus.

FIG. 3 is a flow chart of an exemplary method described herein.

### DETAILED DESCRIPTION

### I. Overview

The embodiments described herein recognize that biomanufacturing is an essential component of biologics production and thus modern medicine.

The Cell Retention Devices (CRDs) play a critical role in establishing a perfusion process. In general, during the perfusion process, CRDs enable the retention of cells in the bioreactor while fresh medium is continuously supplied to the bioreactor, and permeate (e.g., spent media) is continuously pulled out at predetermined rates. Cell retention devices that are currently available in the market (e.g., ATFs and TFFs) are unable to successfully operate large-scale cell (e.g., CHO cell) culture working volumes (e.g., ≥3000 L) with a high perfusion rate (e.g., ≥2 vessel volume per day (VVD)). These cell retention devices have limitations on flux of about 13 Liter/m²/h, and throughput of about 17,500 L/m².

As described herein, in some embodiments, the inventors have shown that HF filters (e.g., TFDF® filters) can be used as a cell retention device for large-scale cell (e.g., CHO cell) culture working volumes (e.g., ≥3000 L) with a high perfusion rate (e.g., ≥2 vessel volume per day (VVD) resulting in higher cell density and titer. As shown herein, the inventors have successfully created a perfusion capable HF filter skid (e.g., TFDF® filter skid) and tested HF filters (e.g., TFDF® filters) with parameters up to 600 Liter/m²/h flux and 70,000 L/m² throughput. In some embodiments, the inventors have discovered that TFDF® comprising systems were suitable for operation at up to 5 bioreactor VVD. In addition, the inventors have found that in some embodiments, the same HF filter (e.g., TFDF® filter) utilized during a perfusion process can also be used for a harvest process, such as but not limited to continuous harvest (CH) processes (e.g., also known as extended harvest processes, etc.). To the inventors' knowledge, this is the first demonstration that such a process can be done with such high pressures and/or volumes in process modes other than harvest modes. In some embodiments, an HF filter can be utilized for perfusion, batch, fed batch, continuous, and/or semi-continuous harvest. In some methods comprising CH, before the end of the production run, the permeate from the HF filter is collected for downstream processing while fresh cell culture media (e.g., comprising metabolites/buffer) is perfused through the bioreactor, while later in the production run fresh cell culture media (e.g., comprising metabolites/buffer) addition is stopped and/or reduced while permeate-draw is still active, leading to a reduction of bioreactor volume. In some embodiments, all cell culture fluid and/or all permeate is processed in a downstream process (DSP).

In some embodiments, as described herein, are systems, apparatuses, and methods which utilize HF filters to achieve higher working volumes (e.g., >3,000 L), higher VVD, increased media exchange rates during perfusion, higher flux, multi-stage use (e.g., one or more perfusion stages and one or more harvest stages), and/or higher throughput, relative to traditional systems, methods and/or apparatus, while maintaining low cell shear rates and/or cell stress levels, thus facilitating culture of relatively sensitive cells (e.g., animal cells, e.g., cells devoid of cell walls) without said cells passing through an associated filter.

In some embodiments, described herein are technologies, such as a multi-purpose assembly and/or system, that are operable with high flow rates, large volumes, any sized bioreactor, and at any stage of a cell culture cycle. As described herein, in some embodiments, such capacity (e.g., suitability for high flow rates, large volumes, any sized bioreactor, and/or use at any stage of a cell culture cycle) is facilitated by the features associated with a hollow fiber filter, and in certain embodiments, a hollow fiber filter operable in a tangential flow mode.

In some embodiments, described herein are technologies, such as a multi-purpose assembly, that can be employed to reduce risk of contamination, reduce consumables attrition rates, reduce the physical footprint associated with biomanufacturing, enable continuous or semi-continuous bioprocessing to purification, and/or simplify the biologics production process.

In certain embodiments, provided herein are technologies that reduce a physical footprint associated with biomanufacturing (e.g., required manufacturing area), for example, a reduction of the necessary footprint by equal to or greater than about 50%. In some embodiments, provided herein are methods systems and apparatus that can reduce the physical footprint associated with biomanufacturing by equal to or greater than about 66%. In some embodiments, a reduction in physical footprint is achieved by utilizing systems and/or assemblies described herein to combine one or more biomanufacturing processes (e.g., perfusion processes and/or harvest processes) into the same physical vessel and/or system, for example, to utilize the same bioreactor for a perfusion process as used for a harvest process (e.g., batch, fed batch, continuous, and/or semi-continuous harvest).

In some embodiments, technologies described herein provide additional automation capabilities. For example, in some embodiments, technologies described herein provide for the avoidance of physical transfer of cell culture fluid from one bioreactor to another, and thus reduce the number of manual processes and increase the potential for process automation.

In some embodiments, technologies described herein provide improved efficiency. For example, in some embodiments, automated processes (e.g. cell-density-triggered phase transitions (e.g., inoculations, filling up of a bioreactor, addition of an inducible agent, etc.,)) can provide manufacturers with little to no cell-waste, process-waste, and/or time-waste. In some embodiments, technologies described herein provide for a reduction in labor/handling effort. For example, in some embodiments, no dedicated harvest device needs to be prepared to receive the cell culture fluid (e.g., culture-broth) produced during the preparatory perfusion processes, as the harvest system and perfusion system are one in the same.

In some embodiments, technologies described herein provide reductions in consumable consumption rates. For example, in some embodiments, a reduction of necessary consumables by equal to or greater than about 50% can be achieved. In some embodiments, technologies described herein provide for a reduction in consumable costs, for example, as no additional cell retention devices (CRDs) are necessary for harvesting (e.g., the same CRD is utilized during perfusion stage(s) and harvest stage(s)), one CRD can be utilized for both processes. In some embodiments, technologies provided herein can include the use of the same CRD during a perfusion process comprising more than one perfusion stage, such as but not limited to, 1, 2, 3, 4, 5, 6, or more than 6 perfusion stages. In some embodiments, a perfusion process comprises a series of perfusion stages (e.g., the seed train). In some embodiments, a series of perfusion stages can comprise an incremental increase in bioreactor volume and/or viable cell density at each subsequent stage. In some embodiments, technologies provided herein can include the use of the same CRD for greater than or equal to about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 days, or greater than 70 days, for greater than or equal to 1, 2, 3, 4, 5, 6, or more than 6 perfusion stages, and for at least one harvest stage. As such, in some embodiments, technologies described herein provide for less consumable storage management effort, and/or less storage footprint.

In some embodiments, technologies described herein provide for less risk of contamination. For example, in some embodiments, less manual applications and the reduction in transfers of cell culture fluid or biologic producing material reduces the potential for contaminant introduction.

In some embodiments, technologies described herein provide for a reduction in the number of CRDs required during a biomanufacturing process, particularly in conditions of biomanufacturing at large scale. For example, in some embodiments a CRD that is utilized during perfusion process can be utilized during a harvest process (e.g., batch, fed batch, continuous, and/or semi-continuous harvest). In some embodiments, the CRD (e.g., HF filter) is already attached during the perfusion process, and is reused during the harvest phase (e.g., perfusion, batch, fed batch, continuous, semi-continuous, bulk harvest, etc.). In some embodiments, a CRD remains attached to the bioreactor during the transition between processes. In some embodiments, the CRD is in “stand-by” mode, for example, cells are still circulating through the lumen of the filter fibers, but no permeate is drawn through the CRD.

The description below provides exemplary implementations of the apparatus, systems, and methods described herein for biomanufacturing. Descriptions and examples of various terms, as used herein, are provided in Section II below.

### II. Exemplary Descriptions of Terms

It is to be understood that the terminology used herein is for the purpose of describing particular embodiments only, and is not intended to be limiting.

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art. Generally, nomenclatures utilized in connection with, and techniques of, chemistry, biochemistry, molecular biology, pharmacology and toxicology are described herein are those well-known and commonly used in the art.

Throughout this disclosure, various aspects are presented in a range format. It should be understood that the description in range format is merely for convenience and brevity and should not be construed as an inflexible limitation on the disclosure. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed in the disclosure. The upper and lower limits of these smaller ranges may independently be included in the smaller ranges, and are also encompassed in the disclosure, subject to any specifically excluded limit in the stated range. Where the stated range includes one or both of the limits, ranges excluding either or both of those included limits are also included in the disclosure. This applies regardless of the breadth of the range.

The headers and subheaders between sections and subsections of this document are included solely for the purpose of improving readability and do not imply that features cannot be combined across sections and subsection. Accordingly, sections and subsections do not describe separate embodiments.

All references cited herein, including patent applications, patent publications, and UniProtKB/Swiss-Prot Accession numbers are herein incorporated by reference in their entirety, as if each individual reference were specifically and individually indicated to be incorporated by reference.

As used herein, the singular forms “a” “an” and “the” are intended to include the plural forms as well, unless the context clearly indicates otherwise. It is also to be understood that the term “and/or” as used herein refers to and encompasses any and all possible combinations of one or more of the associated listed items. It is further to be understood that the terms “includes” “including” “comprises” and/or “comprising” when used herein, specify the presence of stated features, integers, steps, operations, elements, components, and/or units but do not preclude the presence or addition of one or more other features, integers, steps, operations, elements, components, units, and/or groups thereof.

The term “about” as used herein refers to include the usual error range for the respective value readily known. Reference to “about” a value or parameter herein includes (and describes) embodiments that are directed to that value or parameter per se. For example, description referring to “about X” includes description of “X”. In some embodiments, “about” may refer to ±15%, ±10%, ±5%, or ±1% as understood by a person of skill in the art.

The term “amino acid,” as used herein, generally refers to any organic compound that includes an amino group (e.g., —NH2), a carboxyl group (—COOH), and a side chain group (R) which varies based on a specific amino acid. Amino acids can be linked using peptide bonds.

As used herein, the phrase “at least one of,” when used with a list of items, means different combinations of one or more of the listed items may be used and only one of the items in the list may be needed. The item may be a particular object, thing, step, operation, process, or category. In other words, “at least one of” means any combination of items or number of items may be used from the list, but not all of the items in the list may be required. For example, without limitation, “at least one of item A, item B, or item C” or “at least one of item A, item B, and item C” may mean item A; item A and item B; item B; item A, item B, and item C; item B and item C; or item A and C. In some cases, “at least one of item A, item B, or item C” or “at least one of item A, item B, and item C” may mean, but is not limited to, two of item A, one of item B, and ten of item C; four of item B and seven of item C; or some other suitable combination.

The use of the term “or” in the claims is used to mean “and/or” unless explicitly indicated to refer to alternatives only or the alternatives are mutually exclusive, although the disclosure supports a definition that refers to only alternatives and “and/or.” For example, “x, y, and/or z” can refer to “x” alone, “y” alone, “z” alone, “x, y, and z,” “(x and y) or z,” “x or (y and z),” or “x or y or z.” It is specifically contemplated that x, y, or z may be specifically excluded from an embodiment.

The term “biologic” as used herein, generally refers to a biological molecule, such as proteins or chemicals produced by a cell or a virus. The term can mean any molecule, such as but not limited to a protein, peptide, antibody (including antibody derivatives such as but not limited to Fc fusions, scFvs, multispecific antibodies, bispecific antibodies, etc.), nucleic acid, metabolite, antigen, chemical, or biopharmaceutical that is produced by a cell or a virus. The term can also mean adeno-associated viruses (AAVs), e.g. AAV-based gene therapy vectors produced by a cell. In some embodiments, a biologic is and/or can be readily rendered suitable for administration to a subject. In some embodiments, a biologic is purified and/or formulated before it is rendered suitable for administration to a subject.

The terms “biological sample,” “biological specimen,” or “biospecimen” as used herein, generally refers to a specimen taken by sampling so as to be representative of the source of the specimen, typically, from a subject. A biological sample can be representative of an organism as a whole, specific tissue, cell type, or category or sub-category of interest. The biological sample can include a macromolecule. The biological sample can include a small molecule. The biological sample can include a virus. The biological sample can include a cell or derivative of a cell. The biological sample can include an organelle. The biological sample can include a cell nucleus. The biological sample can include a rare cell from a population of cells. The biological sample can include any type of cell, including without limitation prokaryotic cells, eukaryotic cells, bacterial, fungal, plant, mammalian, or other animal cell type, mycoplasmas, normal tissue cells, tumor cells, or any other cell type, whether derived from single cell or multicellular organisms. The biological sample can include a constituent of a cell. The biological sample can include nucleotides (e.g., ssDNA, dsDNA, RNA), organelles, amino acids, peptides, proteins, carbohydrates, glycoproteins, or any combination thereof. The biological sample can include a matrix (e.g., a gel or polymer matrix) comprising a cell or one or more constituents from a cell (e.g., cell bead), such as DNA, RNA, organelles, proteins, or any combination thereof, from the cell. The biological sample may be obtained from a tissue of a subject. The biological sample can include a hardened cell. Such hardened cells may or may not include a cell wall or cell membrane. The biological sample can include one or more constituents of a cell but may not include other constituents of the cell. An example of such constituents may include a nucleus or an organelle. The biological sample may include a live cell. The live cell can be capable of being cultured.

Throughout this specification, unless the context requires otherwise, the words “comprise”, “comprises” and “comprising” will be understood to imply the inclusion of a stated step or element or group of steps or elements but not the exclusion of any other step or element or group of steps or elements. By “consisting of” is meant including, and limited to, whatever follows the phrase “consisting of” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are optional and may or may not be present depending upon whether or not they affect the activity or action of the listed elements.

In addition, as the terms “coupled with”, “in communication” or “communicatively coupled with” or similar words are used herein, one element may be capable of communicating directly, indirectly, or both with another element via one or more wired communications links, one or more wireless communications links, one or more optical communications links, or a combination thereof. In addition, where reference is made to a list of elements (e.g., elements a, b, c), such reference is intended to include any one of the listed elements by itself, any combination of less than all of the listed elements, and/or a combination of all of the listed elements.

As used herein, a “contaminant” may refer to something that is considered undesirable in a cell culture fluid and/or a cell culture media. For example, in certain conditions a contaminant may be but is not limited to, unintended cells and/or viruses, cell culture debris, cell product that is not the target product of therapeutic interest, cell metabolites, etc.

As used herein, an “internal standard,” may refer to something that can be contained (e.g., spiked-in) in the same sample as a target. Internal standards can be used for calibration purposes. Additionally, internal standards can be used in the systems and method described herein.

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present invention. Thus, the appearances of the foregoing phrases in various places throughout this specification are not necessarily all referring to the same embodiment. Furthermore, the particular features, structures, or characteristics may be combined in any suitable manner in one or more embodiments.

As used herein, a “feed stream” is a stream of fluid that leaves a bioreactor, it may comprise cells, cell media, biologics, etc., and it is directed to a filter. In general, a feed stream comprises a lower concentration of cells than a retentate stream. In certain cases, when permeate is not being pulled from a filter, both streams may have the same concentration of cells.

As used herein, the term “harvest permeate stream” comprises fluid that passes through a filter membrane and leaves a filter, it may comprise cell media, biologics, etc. and it is generally directed for further processing. In general, a harvest permeate stream is active during a harvest process and comprises a higher concentration of biologics and lower concentration of cells than a retentate stream.

As used herein, a “model” may include one or more algorithms, one or more mathematical techniques, one or more machine learning algorithms, or a combination thereof.

The term “multi-purpose assembly” as used herein, generally refers to an apparatus comprising at least two flowpaths, wherein each flowpath is operably linked to a pump that is configured to draw a fluid from a permeate stream of the cell retention device at a desired rate.

As used herein, the term “ones” means more than one.

As used herein, the term “plurality” may be 2, 3, 4, 5, 6, 7, 8, 9, 10, or more.

The term “peptide,” as used herein, generally refers to amino acids linked by peptide bonds. Peptides can include amino acid chains between 10 and 50 residues. Peptides can include amino acid chains shorter than 10 residues, including, oligopeptides, dipeptides, tripeptides, and tetrapeptides. Peptides can include chains longer than 50 residues and may be referred to as “polypeptides” or “proteins.”

As used herein, the term “perfusion permeate stream” comprises fluid that passes through a filter membrane and leaves a filter, it may comprise cell media, biologics, etc. and it is generally directed for further processing and/or disposal. In general, a perfusion permeate stream is active during a perfusion process and comprises a higher concentration of cell media and lower concentration of biologics than a harvest permeate stream.

As used herein, the term “permeate” comprises a fluid that passes through a filter membrane and leaves a filter.

The terms “protein” or “polypeptide” or “peptide” may be used interchangeably herein and generally refer to a molecule including at least three amino acid residues. Proteins can include polymer chains made of amino acid sequences linked together by peptide bonds. Proteins may be digested in preparation for mass spectrometry using trypsin digestion protocols. Proteins may be digested using other proteases in preparation for mass spectrometry if access is limited to cleavage sites.

As used herein, the term “retentate stream” comprises fluid that leaves a filter without passing through a filter membrane (e.g., it passes through a filter lumen), it may comprise cells, cell media, biologics, etc., and it is generally directed to a bioreactor. In general, a retentate stream comprises a higher concentration of cells than a feed stream. In certain cases, when permeate is not being pulled from a filter, both streams may have the same concentration of cells.

The term “sample,” as used herein, generally refers to a sample from a subject of interest and may include a biological sample of a subject. The sample may include a cell sample. The sample may include a cell line or cell culture sample. The sample can include one or more cells. The sample can include one or more microbes. The sample may include a nucleic acid sample or protein sample. The sample may also include a carbohydrate sample or a lipid sample. The sample may be derived from another sample. The sample may include a tissue sample, such as a biopsy, core biopsy, needle aspirate, or fine needle aspirate. The sample may include a fluid sample, such as a blood sample, urine sample, or saliva sample. The sample may include a skin sample. The sample may include a cheek swab. The sample may include a plasma or serum sample. The sample may include a cell-free or cell free sample. A cell-free sample may include extracellular polynucleotides. The sample may originate from blood, plasma, serum, urine, saliva, mucosal excretions, sputum, stool, or tears. The sample may originate from red blood cells or white blood cells. The sample may originate from feces, spinal fluid, CNS fluid, gastric fluid, amniotic fluid, cyst fluid, peritoneal fluid, marrow, bile, other body fluids, tissue obtained from a biopsy, skin, or hair.

As used herein, the term “set of” means one or more. For example, a set of items includes one or more items.

The term “sequence,” as used herein, generally refers to a biological sequence including one-dimensional monomers that can be assembled to generate a polymer. Non-limiting examples of sequences include nucleotide sequences (e.g., ssDNA, dsDNA, and RNA), amino acid sequences (e.g., proteins, peptides, and polypeptides), and carbohydrates (e.g., compounds including Cₘ (H₂O)ₙ).

The term “subject,” as used herein, generally refers to an animal, such as a mammal (e.g., human) or avian (e.g., bird), or other organism, such as a plant. For example, the subject can include a vertebrate, a mammal, a rodent (e.g., a mouse), a primate, a simian, or a human. Animals may include, but are not limited to, farm animals, sport animals, and pets. A subject can include a healthy or asymptomatic individual, an individual that has or is suspected of having a disease (e.g., cancer) or a pre-disposition to the disease, and/or an individual that needs therapy or suspected of needing therapy. A subject can be a patient. A subject can include a microorganism or microbe (e.g., bacteria, fungi, archaea, viruses).

As used herein, “substantially” means sufficient to work for the intended purpose. The term “substantially” thus allows for minor, insignificant variations from an absolute or perfect state, dimension, measurement, result, or the like such as would be expected by a person of ordinary skill in the field but that do not appreciably affect overall performance. When used with respect to numerical values or parameters or characteristics that can be expressed as numerical values, “substantially” means within ten percent.

The ensuing description provides exemplary embodiments only, and is not intended to limit the scope, applicability or configuration of the disclosure. Rather, the ensuing description of the preferred exemplary embodiments will provide those skilled in the art with an enabling description for implementing various embodiments. It is understood that various changes may be made in the function and arrangement of elements without departing from the spirit and scope as set forth in the appended claims.

Specific details are given in the following description to provide a thorough understanding of the embodiments. However, it will be understood that the embodiments may be practiced without these specific details. For example, circuits, systems, networks, processes, and other components may be shown as components in block diagram form in order not to obscure the embodiments in unnecessary detail. In other instances, well-known circuits, processes, algorithms, structures, and techniques may be shown without unnecessary detail in order to avoid obscuring the embodiments.

### III. Overview of a Multi-Purpose Assembly

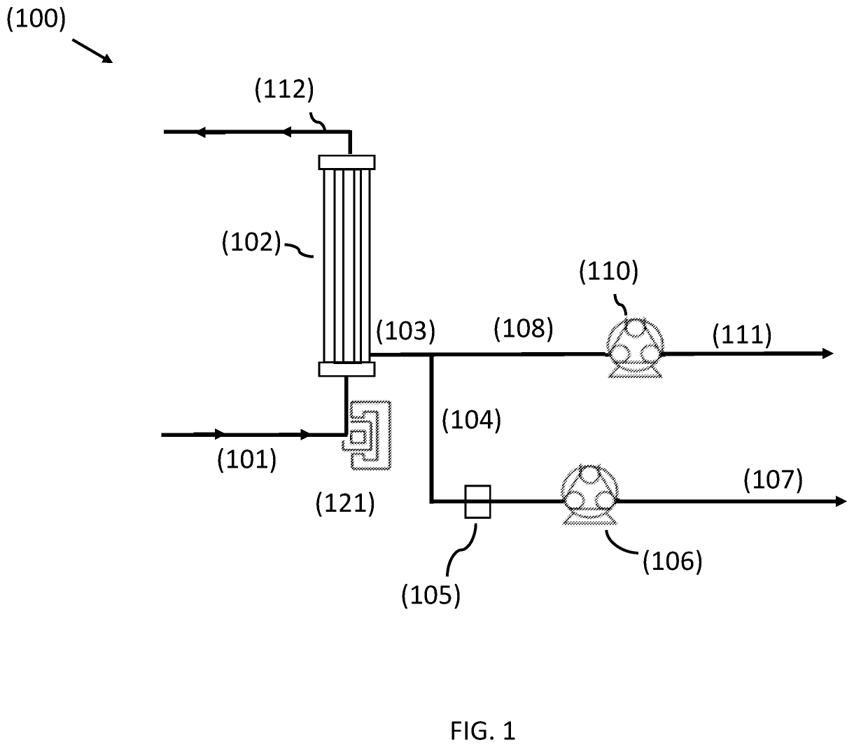

Referring to FIG. 1, illustrated is a non-limiting schematic diagram of an exemplary multi-purpose assembly 100 for the improved production of biologics. The assembly comprises a feed stream conduit 101 operably connected to an inlet of a cell retention device such as a Hollow Fiber (HF) filter 102. In certain embodiments, a feed stream conduit is operable with an optional feed pump 121. In some embodiments, a HF filter is operable for tangential flow. In some embodiments, a HF filter is a tangential flow depth filtration® (TFDF®) filter. The assembly is operable to facilitate fluid (e.g., fluid comprising cell culture media, cells, biologics, etc.) entering the HF filter while allowing appropriate fluid components (e.g., those sufficiently sized to do so) to pass through a filter membrane to leave the HF filter as permeate 103. The HF filter is operable such that fluid that does not pass through a filter membrane can leave the HF filter as retentate 112. Permeate fluid can be pulled through the HF filter through the action of two or more pumps to enter two or more flowpaths. Under certain operating conditions, such as operating in a perfusion process, a first pump 106 is operable to pull a permeate 103 into a first flowpath 104 to form a perfusion permeate 107. The first pump 106 is operably in communication with a sensor 105 operable for accurate measurement of a fluid flow rate, such that the first pump 106 is operable for maintaining a fluid flow rate suitable for a perfusion process, for example, wherein a perfusion permeate 107 from the HF filter is pulled at a consistent and/or desirable rate. Under certain operating conditions, such as operating in a harvest process, a second pump 110 is operable to pull a permeate 103 into a second flowpath 108 to form a harvest permeate 111. The second pump 110 and harvest permeate 111 are optionally operable for communication with one or more sensors and/or fluid communication with one or more downstream filtering units and/or processing units suitable for maintaining a harvest process. In general, the second flowpath 108 and second pump 110 draw permeate at a higher fluid flow rate than a first flowpath 104 and a first pump 106.

In certain embodiments, a multi-purpose assembly is operable for one or more perfusion processes (such as but not limited to, 1, 2, 3, 4, 5, 6, or more than 6 perfusion processes) and one or more harvest processes (e.g., a harvest process and/or an additional process following the onset of a harvest process). In certain embodiments, a multi-purpose assembly is operable for one or more perfusion processes, one or more in-between processes (e.g., a process between one or more processes, in such a process, permeate may or may not be drawn while fluid flow may or may not be maintained), and one or more harvest processes (e.g., an additional process following the onset of a harvest process). In some embodiments, such a combination of processes is referred to as a combined process.

In certain embodiments, the multi-purpose assembly is connected to a turndown bioreactor. In certain embodiments, the turndown ratio is from about 30:1 to about 2:1. In certain embodiments, the turndown ratio is from about 20:1 to about 5:1. In certain embodiments, the turndown ratio is from about 10:1 to about 5:1. In certain embodiments, one or more of the multi-purpose assemblies used in the methods of the present disclosure are connected to bioreactor that has a turn down ratio of about 10:1. In certain embodiments, one or more of the multi-purpose assemblies used in the methods of the present disclosure are connected to bioreactor that has a turn down ratio of about 5:1.

In certain embodiments, more than one multi-purpose assembly can be utilized in one system. In certain embodiments, one, two, three, four, or five multi-purpose assemblies are utilized in one system. In certain embodiments, when more than one multi-purpose assembly is utilized in one system, the assemblies are utilized sequentially, and/or in tandem. In certain embodiments, utilization of more than one multi-purpose assembly in a system comprising a bioreactor can allow for increased bioreactor volumes relative to a system with only one multi-purpose assembly.

In certain embodiments, exemplary operating parameters for three target throughputs achievable using a multi-purpose assembly and/or system comprising the same are provided in Table 1.

TABLE 1
Exemplary Perfusion Process Operating Parameters

| Phase | Parameter | Target 1 | Target 2 | Target 3 |
|---|---|---|---|---|
| Perfusion | Throughput (L/m²) | >10,000 | >25,000 | >30,000 |
| process | Operating | 20-40 | 30-38 | 35-38 |
| | Temperature (° C.) | | | |
| | Feed flow rate | 0.8-2.2 | 0.8-2.0 | 1-1.8 |
| | (L/fiber/min) | | | |
| | Filter Flux (LMH) | 50-800 | 100-600 | 100-500 |
| | PCV (%) | 8-40 | 12-35 | 12-35 |
| | VCD (×10\hat{ }6 | >50 | >55 | >55 |
| | cells/mL) | | | |
| | VVD | 0.1-5.0 | 1-4.3 | 1.5-4.3 |
| | Process Duration | ≥5 | ≥6 | ≥6 |
| | (Day) | | | |
| | Shear Rate (s−1) | <5000 | <3500 | <2500 |

III.A. Cell Retention Device (CRD)

In certain embodiments, a CRD is a filter suitable for both perfusion and harvest processes. In certain embodiments, a CRD is a Hollow Fiber (HF) filter. In certain embodiments, a HF filter is operable for tangential flow. In certain embodiments, a HF filter is a 3E-NF20A, 3E-NF40A, 3E-NF60, 3E-NF80A, 3E-NF90A (3E Memtech Pte Ltd.), De.mem NF (De.mem Limited), dNF80, dNF40 (NX Filtration), HFW100 (Pentair X-Flow™) NUF N80 (Ochemate®), or tangential flow depth filtration® (TFDF®; Repligen® Inc.) filter. In certain embodiments, a HF filter is a tangential flow depth filtration® (TFDF®) filter. In certain embodiments, a HF filter is comprised of PES/PVDF, PEI based TFC, PES-PEM, mPES, and/or Polyamide based TFC.

In some embodiments, a HF filter is constructed of and/or comprises a synthetic polymer. In some embodiments, a HF filter is constructed of and/or comprises polypropylene, and/or polyethylene terephthalate. In some embodiments a HF filter is constructed of and/or comprises a modified polyethersulfone, polyethersulfone, mixed cellulose ester, and/or polysulfone.

In certain embodiments, a HF filter has a filter surface area ranging from about 3 to about 6240 cm². In certain embodiments, a HF filter has a filter surface area of about 3 cm², about 30 cm², about 50 cm², about 150 cm², about 450 cm², about 750 cm², about 1,500 cm², about 2,100 cm², about 6,000 cm², or more than about 6,000 cm². In certain embodiments, a HF filter has a filter surface area of about 150, 300, 450, 600, 750, 900, 1,050, 1,200, 1,350, 1,500, 1,650, 1,800, 1,950, 2,100, 2,250, 2,400, 2,550, 2,700, 2,850, 3,000, 3,150, 3,300, 3,450, 3,600, 3,750, 3,900, 4,050, 4,200, 4,350, 4,500, 4,650, 4,800, 4,950, 5,100, 5,250, 5,400, 5,550, 5,850, 6,000, 6,150, 6,300, 6,450, 6,600, 6,750, 6,900, 7,050, 7,200, 7,350, 7,500, 7,650, 7,800, 7,950, 8,100, 8,250, 8,400, 8,550, 8,700, 8,850, 9,000, or greater than 9,000 cm².

In certain embodiments, a HF filter comprises one or more fibers. In certain embodiments, fibers have a lumen through which a feed stream can flow, surrounded by a membrane layer through which permeate can pass. In certain embodiments, permeate fluid that is drawn through a membrane layer can leave a HF filter as permeate (see e.g., FIG. 1, permeate 103). In certain embodiments, a tube is 20, 36, or 108 cm in length. In certain embodiments, a tube is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 121, 122, 123, 124, 125, 126, 127, 128, 129, 130, or greater than 130 cm in length.

In certain embodiments, a HF filter comprises more than 1 fiber. In some embodiments, a HF filter comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 or more fibers.

In some embodiments, a HF filter has an average pore size diameter of between about 0.65 μm to about 8 μm. In some embodiments, the HF filter has an average pore size diameter of about 2 μm to about 5 μm. In some embodiments, the HF filter has an average pore size diameter of about 2 μm. In certain embodiments, a HF filter may have an average pore size of about 0.5, 1, 1.5, 2, 2.5, 3, 3.5, 4, 4.5, 5, 5.5, 6, 6.5, 7, 7.5, 8, 8.5, 9, 9.5, or 10 μm or any range derivable therein. In certain embodiments, a pore structure is anisotropic. In certain embodiments, a pore structure is not anisotropic. In certain embodiments, a pore structure is isotropic.

In certain embodiments, a HF filter filtrate channel comprises a filter having about a 2 mm internal diameter lumen therethrough. In certain embodiments, a HF filter filtrate channel comprises a filter having about a 4.6 mm internal diameter lumen therethrough. In certain embodiments a TFDF® filter filtrate channel comprises a filter having about a 0.5, 1, 1.5, 2, 2.5, 3, 3.5, 4, 4.5, 5, 5.5, 6, 6.5, 7, 7.5, 8, 8.5, 9, 9.5, or 10 mm or any range derivable therein, internal diameter lumen therethrough. In certain embodiments, a HF filter filtrate channel comprises a filter having about a 0.4 mm to about 1.5 mm lumen therethrough. In certain embodiments, a HF filter filtrate channel does not have less than about a 0.4 mm lumen therethrough.

In certain embodiments, a HF filter has a wall thickness of about 0.05 mm to about 0.075, 0.1, 0.125, 0.15, 0.175, 0.2, 0.225, 0.25, 0.275, 0.3, 0.325, 0.35, 0.375, 0.4, 0.425, 0.45, 0.475, 0.5 mm, or any range derivable therein. In certain embodiments, a HF filter has a wall thickness of about 1 mm to about 1.25, 1.5, 1.75, 2, 2.25, 2.5, 2.75, 3, 3.25, 3.5, 3.75, 4, 4.25, 4.5, 4.75, 5, 5.25, 5.5, 5.75, 6, 6.25, 6.5, 6.75, 7, 7.25, 7.5 mm, or any range derivable therein. In certain embodiments, a HF filter has a wall thickness of about 5 mm.

In certain embodiments, a permeate can flow through a HF filter at a rate of flow greater than about 0.1, 0.5, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0, 10.5, 11.0, 11.5, 12.0, 12.5, 13.0, 13.5, 14.0, 14.5, 15.0, 15.5, 16.0, 16.5, 17.0, 17.5, 18.0, 18.5, 19.0, 19.5, or 20.0 liters per minute (LPM). In certain embodiments, a permeate can flow through a HF filter at a rate of flow between about 0 LPM and/or about 0.1 LPM to about 20 LPM, or any range derivable therein. In certain embodiments, a permeate can flow through a HF filter at a rate of flow between about 0 LPM and/or about 0.1 LPM to about 5 LPM. In certain embodiments, a permeate can flow through a HF filter at a rate of flow between about 0 LPM and/or about 0.1 LPM to about 8 LPM. In certain embodiments, a permeate can flow through a HF filter at a rate of flow between about 0 LPM and/or about 0.1 LPM to about 10 LPM. In certain embodiments, a permeate can flow through a HF filter at a rate of flow between about 0 LPM and/or about 0.1 LPM to about 13 LPM. In certain embodiments, a permeate can flow through a HF filter at a rate of flow between about 0 LPM and/or about 0.1 LPM to about 15 LPM. In certain embodiments, a permeate can flow through a HF filter at a rate of flow between about 0 LPM and/or about 0.1 LPM to about 18 LPM.

In certain embodiments, a permeate can flow through a HF filter while maintaining a shear rate of less than about 10,000, 9,000, 8,000, 7,000, 6,000, 5,000, 4,500, 4,000, 3,500, 3,000, 2,500, 2,000, 1,500, 1,000, or 500 s−1.

In certain embodiments, a HF filter has a throughput greater than about 1,000, 2,000, 3,000, 4,000, 5,000, 6,000, 7,000, 8,000, 9,000, 10,000, 11,000, 12,000, 13,000, 14,000, 15,000, 16,000, 17,000, 18,000, 19,000, 20,000, 21,000, 22,000, 23,000, 24,000, 25,000, 26,000, 27,000, 28,000, 29,000, 30,000, 31,000, 32,000, 33,000, 34,000, 35,000, 36,000, 37,000, 38,000, 39,000, 40,000, 41,000, 42,000, 43,000, 44,000, 45,000, 46,000, 47,000, 48,000, 49,000, 50,000, 51,000, 52,000, 53,000, 54,000, 55,000, 56,000, 57,000, 58,000, 59,000, 60,000, 61,000, 62,000, 63,000, 64,000, 65,000, 66,000, 67,000, 68,000, 69,000, 70,000, 71,000, 72,000, 73,000, 74,000, 75,000, 76,000, 77,000, 78,000, 79,000, or 80,000 liters per meter square (L/m²). In some embodiments, a HF filter has a throughput greater than about 10,000 L/m². In some embodiments, a HF filter has a throughput greater than about 25,000 L/m². In some embodiments, a HF filter has a throughput greater than about 30,000 L/m². In some embodiments, a HF filter has a throughput greater than about 40,000 L/m². In some embodiments, a HF filter has a throughput equal to between about 5,000 to about 35,000 L/m², or any range derivable therein.

In certain embodiments, a HF filter has a filter flux rate of between about 20 and about 800 liters per square meter per hour (LMH). In certain embodiments, a HF filter has a filter flux rate of between about 100 and about 600 LMH. In certain embodiments, a HF filter has a filter flux rate of between about 300 and about 600 LMH. In certain embodiments, a HF filter has a filter flux rate of between about 400 and about 600 LMH. In certain embodiments, a HF filter has a filter flux rate of between about 50 and about 100, about 50 and about 150, about 50 and about 200, about 50 and about 250, about 50 and about 300, about 50 and about 350, about 50 and about 400, about 50 and about 450, about 50 and about 500, about 50 and about 550, about 50 and about 600, about 50 and about 650, about 50 and about 700, about 50 and about 750, about 50 and about 800, about 50 and about 850, or about 50 to about 900 LMH.

In some embodiments, a multi-purpose filter assembly is operable for maintaining a viable cell density (VCD) of greater than about 1×10⁶ cells/mL. In some embodiments, a multi-purpose filter assembly is operable for maintaining a viable cell density (VCD) of greater than about 10×10⁶ cells/mL. In some embodiments, a multi-purpose filter assembly is operable for maintaining a viable cell density (VCD) of greater than about 25×10⁶ cells/mL. In some embodiments, a multi-purpose filter assembly is operable for maintaining a VCD of greater than about 50×10⁶ cells/mL. In some embodiments, a multi-purpose filter assembly is operable for maintaining a VCD of greater than about 75×10⁶ cells/mL. In some embodiments, a multi-purpose filter assembly is operable for maintaining a VCD of greater than about 10×10⁷ cells/mL. In some embodiments, a multi-purpose filter assembly is operable for maintaining a VCD of greater than about 25×10⁷ cells/mL. In some embodiments, a multi-purpose filter assembly is operable for maintaining a VCD of greater than about 50×10⁷ cells/mL. In some embodiments, a multi-purpose filter assembly is operable for maintaining a VCD of greater than about 75×10⁷ cells/mL. In some embodiments, a multi-purpose filter assembly is operable for maintaining a VCD of greater than about 10×10 8 cells/mL. In some embodiments, a multi-purpose filter assembly is operable for maintaining a VCD of greater than about 25×10 8 cells/mL. In some embodiments, a multi-purpose filter assembly is operable for maintaining a VCD of greater than about 50×10 8 cells/mL. In some embodiments, a VCD is the VCD in a bioreactor fluid volume. In some embodiments, a VCD is the VCD in a retentate stream of a CRD.

III.B. Pumps

In some embodiments, assemblies, systems, and/or methods described herein comprise or comprise the use of one or more pumps.

In certain embodiments, a pump may be operable for pushing or pulling fluid through a suitable conduit. In certain embodiments, a pump can be but is not limited to, a pump selected from the group consisting of peristaltic, centrifugal, magnetic drive, positive displacement, membrane, pressure-based, Quantex™ (e.g., positive displacement rotary pumps), gear, diaphragm, syringe, and piston pumps. In certain embodiments, a pump is a magnetic drive pump. In certain embodiments, a pump is a peristaltic pump. In certain embodiments, one or more pumps are suitable for generating alternating tangential flow. In certain embodiments, one or more pumps are not utilized and/or operable for generation of alternating tangential flow.

In certain embodiments, a pump is operable to maintain a desired flow rate. In certain embodiments, a pump is in communication with one or more sensors which can direct a pump to increase or decrease pumping force to alter the flow rate as desired. In some embodiments, a flow rate is measured in liters per fiber per minute (L/fiber/min). In some embodiments, a flow rate is or is about 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, or 5 L/fiber/min. In some embodiments, a flow rate is about 0.5 to about 2.5 L/fiber/min. In some embodiments, a flow rate is about 0.8 to about 2.2 L/fiber/min. In some embodiments, a flow rate is about 0.8 to about 2.0 L/fiber/min. In some embodiments, a flow rate is about 1 to about 1.8 L/fiber/min.

In some embodiments, a flow rate is determined as a function of a set vessel volume per day (VVD). In some embodiments, a pump can maintain a flow rate that equates to about 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, or 5 VVD. In some embodiments, a pump can maintain a flow rate of about 2 VVD. In some embodiments, a pump can maintain a flow rate of about 3 VVD. In some embodiments, a pump can maintain a flow rate of about 4 VVD. In some embodiments, a pump can maintain a flow rate of about 5 VVD. In some embodiments, a pump can maintain a flow rate of greater than about 5 VVD. In some embodiments, a pump can maintain a higher VVD rate when a relatively large filter is utilized in conjunction with a relatively small bioreactor.

In some embodiments, a system may comprise more than one multi-purpose assembly. In some embodiments, wherein a system comprises more than one multi-purpose assembly, a VVD rate can be amplified accordingly, for example but not limited to, a VVD of about 5, 6, 7, 8, 9, 10, or greater than 10.

In some embodiments, a pump is operable for accurately facilitating fluid flow at a rate of flow greater than about 0.1, 0.5, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0, 10.5, 11.0, 11.5, 12.0, 12.5, 13.0, 13.5, 14.0, 14.5, 15.0, 15.5, 16.0, 16.5, 17.0, 17.5, 18.0, 18.5, 19.0, 19.5, or 20.0 liters per minute (LPM). In some embodiments, a pump is operable for accurately facilitating fluid flow at a rate of flow greater than about 0.1, 0.5, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0, 10.5, 11.0, 11.5, 12.0, 12.5, 13.0, 13.5, 14.0, 14.5, 15.0, 15.5, 16.0, 16.5, 17.0, 17.5, 18.0, 18.5, 19.0, 19.5, 20.0, 20.5, 21, 21.5, 22, 22.5, 23, 23.5, 24, 24.5, 25, 25.5, 26, 26.5, 27, 27.5, 28, 28.5, 29, 29.5, 30, 30.5, 31, 31.5, 32, 32.5, 33, 33.5, 34, 34.5, 35, 35.5, 36, 36.5, 37, 37.5, 38, 38.5, 39, 39.5, 40, 40.5, 41, 41.5, 42, 42.5, 43, 43.5, 44, 44.5, 45, 45.5, 46, 46.5, 47, 47.5, 48, 48.5, 49, 49.5, 50, 50.5, 51, 51.5, 52, 52.5, 53, 53.5, 54, 54.5, 55, 55.5, 56, 56.5, 57, 57.5, 58, 58.5, 59, 59.5, 60, 60.5, 61, 61.5, 62, 62.5, 63, 63.5, 64, 64.5, 65, 65.5, 66, 66.5, 67, 67.5, 68, 68.5, 69, 69.5, 70, 70.5, 71, 71.5, 72, 72.5, 73, 73.5, 74, 74.5, 75, 75.5, 76, 76.5, 77, 77.5, 78, 78.5, 79, 79.5, or 80 LPM, or any range derivable therein.

In some embodiments, a pump is operable for accurately facilitating fluid flow at a rate of flow ranging from about 0 LPM and/or about 0.1 LPM to about 20 LPM, or any range derivable therein. In some embodiments, a pump is operable for accurately facilitating fluid flow at a rate of flow ranging from about 0 LPM and/or about 0.1 LPM to about 5 LPM. In some embodiments, a pump is operable for accurately facilitating fluid flow at a rate of flow ranging from about 0 LPM and/or about 0.1 LPM to about 8 LPM. In some embodiments, a pump is operable for accurately facilitating fluid flow at a rate of flow ranging from about 0 LPM and/or about 0.1 LPM to about 10 LPM. In some embodiments, a pump is operable for accurately facilitating fluid flow at a rate of flow ranging from about 0 LPM and/or about 0.1 LPM to about 13 LPM. In some embodiments, a pump is operable for accurately facilitating fluid flow at a rate of flow ranging from about 0 LPM and/or about 0.1 LPM to about 15 LPM. In some embodiments, a pump is operable for accurately facilitating fluid flow at a rate of flow ranging from about 0 LPM and/or about 0.1 LPM to about 18 LPM.

In some embodiments, a first flowpath pump is operable for accurately facilitating fluid flow at a rate that is less than a second flowpath pump. In some embodiments, a pump associated with a perfusion permeate flowpath is operable for accurately facilitating fluid flow at a rate that is less than a pump associated with a harvest permeate flowpath.

In some embodiments, the first flowpath pump associated with an accurate sensor is suitable for accurate control of a fluid flow rate for a first flowpath. In some embodiments, the first flowpath pump associated with an accurate sensor is suitable for accurate control of a fluid flow rate for the first flowpath, wherein the control is more accurate than the pump utilized to induce flow in a second flowpath.

In some embodiments, a pump may have any sized heading. In some embodiments, a pump heading is sized according to a desired rate of flow, tubing inner diameter, and/or flow accuracy requirement. In some embodiments, tubing in a flowpath may be of any type suitable for cell culture. In some embodiments, tubing may be autoclavable. In some embodiments, tubing is suitable for food grade quality and/or good manufacturing purposes quality. In some embodiments, tubing comprises silicone.

In some embodiments, a pump is operable for maintaining a packed cell volume (PCV) percentage of about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50%, or any range derivable therein. In some embodiments, a pump is operable for maintain a PCV percentage of about 2 to about 40%, about 8 to about 40%, about 12 to about 35%, or about 15 to about 30%.

III.C. Sensors

A multi-purpose assembly comprises at least one sensor suitable for accurate measurement of a fluid flow rate for a first flowpath (see e.g., FIG. 1, flowpath 104). In some embodiments, such a sensor is in communication with a first flowpath pump. In some embodiments, such a sensor is in communication with a second flowpath pump.

In some embodiments, a sensor is operable for accurately measuring fluid flow at a rate of flow greater than about 0.01, 0.1, 0.5, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0, 10.5, 11.0, 11.5, 12.0, 12.5, 13.0, 13.5, 14.0, 14.5, 15.0, 15.5, 16.0, 16.5, 17.0, 17.5, 18.0, 18.5, 19.0, 19.5, or 20.0 liters per minute (LPM).

In some embodiments, a sensor is operable for accurately measuring fluid flow at a rate of flow ranging from about 0 LPM and/or about 0.1 LPM to about 20 LPM, or any range derivable therein. In some embodiments, a sensor is operable for accurately measuring fluid flow at a rate of flow ranging from about 0 LPM and/or about 0.1 LPM to about 5 LPM. In some embodiments, a sensor is operable for accurately measuring fluid flow at a rate of flow ranging from about 0 LPM and/or about 0.1 LPM to about 8 LPM. In some embodiments, a sensor is operable for accurately measuring fluid flow at a rate of flow ranging from about 0 LPM and/or about 0.1 LPM to about 10 LPM. In some embodiments, a sensor is operable for accurately measuring fluid flow at a rate of flow ranging from about 0 LPM and/or about 0.1 LPM to about 13 LPM. In some embodiments, a sensor is operable for accurately measuring fluid flow at a rate of flow ranging from about 0 LPM and/or about 0.1 LPM to about 15 LPM. In some embodiments, a sensor is operable for accurately measuring fluid flow at a rate of flow ranging from about 0 LPM and/or about 0.1 LPM to about 18 LPM.

In some embodiments, the at least one sensor suitable for accurate measurement of a fluid flow rate for a first flowpath is more accurate than one or more additional sensors utilized to measure flow rate in a second flowpath.

In some embodiments, a sensor is operable for communication with a transmitter. In some embodiments, a transmitter is operable for communication with one or more pumps, one or more control units, and/or one or more human machine interfaces.

III.D. Flowpaths

As described herein, in most embodiments, a multi-purpose assembly comprises at least two flowpaths. In certain embodiments, a multi-purpose assembly comprises 2 flowpaths. In certain embodiments, a multi-purpose assembly comprises more than 2 flowpaths, for example but not limited to, 3, 4, 5, 6, 7, 8, 9, 10, or more than 10 flowpaths, or any range derivable therein. In some embodiments, flowpaths in addition to the first two flowpaths may be specialized to be operable for certain processes (e.g., certain perfusion and/or harvest processes), or may be generalized to be operable for any process for which a flowpath is needed.

In certain embodiments, a first flow path is a perfusion permeate flow path (see e.g., FIG. 1, first flowpath 104 and perfusion permeate 107). In some embodiments, a first flow path comprises tubing with an inner diameter (ID) of ½″, ⅜″, ¼″, or ⅛″. In certain embodiments, a first flow path comprises tubing with ⅜″ ID.

In certain embodiments, a second flow path is a harvest permeate flow path (see e.g., FIG. 1, flowpath 108 and harvest permeate 111). In some embodiments, a second flow path comprises tubing with an inner diameter (ID) of ½″, ⅜″, ¼″, or ⅛″. In certain embodiments, a second flow path comprises tubing with ½″ ID.

In certain embodiments, a multi-purpose assembly comprising a first flowpath and pump is operable to draw a flow rate from the HF filter at about 0 to about 3 liters per fiber per minute (L/fiber/min). In some embodiments, a first flowpath and pump are operable to draw a flow rate from the HF filter of about 0.8 to about 2.2 L/fiber/min. In some embodiments, a first flowpath and pump are operable to draw a flow rate from the HF filter of about 0.8 to about 2.0 L/fiber/min. In some embodiments, a first flowpath and pump are operable to draw a flow rate from the HF filter of about 1.0 to about 1.8 L/fiber/min.

In certain embodiments, a multi-purpose assembly comprising a second flowpath and pump is operable to draw a flow rate from the HF filter at about 0 to about 4 liters per fiber per minute (L/fiber/min). In some embodiments, a second flowpath and pump are operable to draw a flow rate from the HF filter of about 1 to about 2.5 L/fiber/min. In some embodiments, a second flowpath and pump are operable to draw a flow rate from the HF filter of about 1 to about 3.0 L/fiber/min. In some embodiments, a second flowpath and pump are operable to draw a flow rate from the HF filter of about 1.5 to about 2 L/fiber/min.

In some embodiments, a multi-purpose assembly comprising a second flowpath and pump is operable to draw fluid from the HF filter at a flow rate of between about 0.01 LPM to about 80 LPM, between about 0.01 LPM to about 60 LPM, between about 0.01 LPM to about LPM, and/or between about 0.01 LPM to about 20 LPM.

In some embodiments, a multi-purpose assembly comprising a second flowpath and pump is operable to draw fluids from the HF filter at a throughput greater than or equal to about 1,000 liters per square meter (L/m²) to about 80,000 L/m², about 10,000 liters per square meter (L/m²) to about 50,000 L/m², and/or about 10,000 liters per square meter (L/m²) to about 70,000 L/m². In some embodiments, a multi-purpose assembly comprising a second flowpath and pump is operable to draw fluids from the HF filter at a throughput greater than or equal to about 4,000 liters per square meter (L/m²). In some embodiments, a multi-purpose assembly comprising a second flowpath and pump is operable to draw fluids from the HF filter at a throughput greater than or equal to about 40,000 liters per square meter (L/m²).

In some embodiments, a first flowpath has an accuracy requirement of about 1%. In some embodiments, a first flowpath has an accuracy requirement of about 2%. In some embodiments, a second flowpath has an accuracy requirement of about 3%. In some embodiments, a second flowpath has an accuracy requirement of about 4%. The accuracy requirement may be a flow accuracy requirement.

In some embodiments, a first and a second flowpath are joined to a primary permeate flowpath (see e.g., FIG. 1, flowpath 103, and first flowpath 104 and second flowpath 108). In some embodiments, the first flowpath and second flowpath of the multi-purpose assembly are connected by any suitable means, such as but not limited to a T connector, a Y connector, or a valve. In some embodiments, an optional clamp (e.g., a pinch clamp) may be operable for directing flow to the first or second flowpath. In some embodiments, the opening and closing of a valve may be automatized.

In some embodiments, a fluid flow rate through a first flowpath is lower than a fluid flow rate through a second flowpath. In some embodiments, a fluid flow rate through a perfusion flowpath is lower than a fluid flow rate through a harvest flowpath.

III.E. Filters

In some embodiments, a multi-purpose assembly may comprise one or more additional filters (e.g., clarification filter). In some embodiments, an additional filter is affixed to a first flowpath. In some embodiments, an additional filter is affixed to a second flowpath. An additional filter may be configured to, but is not limited to, remove contaminates, selectively remove and/or harvest one or more biologic, and/or to ensure closed system sterility.

III.F. Control Units and Human Machine Interface

In some embodiments, provided herein are multi-purpose assemblies that can be operably connected to at least one data processor, at least one control unit, and/or at least one human machine interface (HMI). In some embodiments, a control unit and an HMI can be used to monitor and/or control any adjustable parameters associated with a multi-purpose assembly, such as but not limited to, VVD, flow rate, VCD, pump rate, sensor accuracy, flowpath utilization, etc. In some embodiments, a control unit and an HMI can be used to control additional parameters associated with a system comprising a multi-purpose assembly, such as but not limited to, temperature, fresh media input rates, bleed rates, inducible agent addition rates, oxygen levels, CO₂ levels, metabolite levels, etc.

In some embodiments, customized control strategies are programmed, integrated and/or displayed using a HMI. In some embodiments, any physical components and/or apparatus described herein may be operable for and/or in communication with a HMI.

### IV. Overview of a System Comprising a Multi-Purpose Assembly

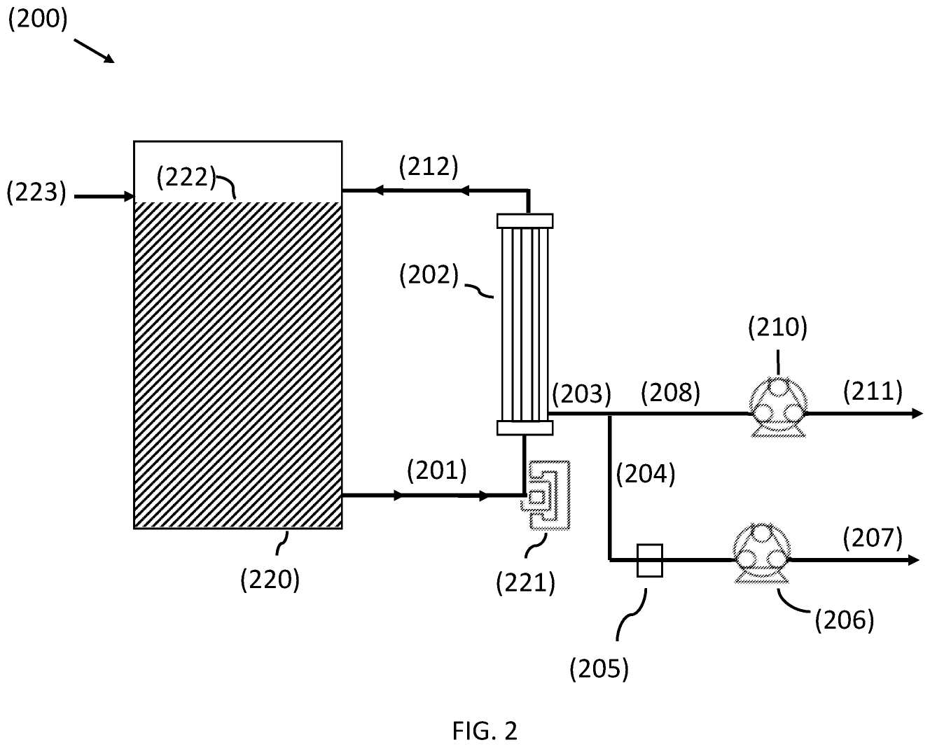

Referring to FIG. 2, illustrated is a non-limiting schematic diagram of an exemplary bioreactor system 200 comprising a multi-purpose assembly (see e.g., FIG. 1, 100) for the improved production of biologics. The system comprises a bioreactor 220, which is operably connected to an optional inlet port (e.g., fresh media input port) 223, an output port (e.g., feed stream) 201, and an input (e.g., retentate) port 212. The system may comprise a bioreactor fluid volume 222. The system comprises a feed stream conduit 201 operably connected to the output port 201 and a inlet of a cell retention device such as a Hollow Fiber (HF) filter 202. In some embodiments, such a HF filter is operable for tangential flow. In some embodiments, such an HF filter is a tangential flow depth filtration® (TFDF®) filter. The output port 201 can optionally include a pump 221 to facilitate fluid flow from the bioreactor to the HF filter 202. The system is operable to facilitate fluid (e.g., fluid comprising cell culture media, cells, biologics, etc.) entering the HF filter while allowing appropriate fluid components (e.g., those sufficiently sized to do so) to pass through a filter membrane to leave the HF filter as permeate 203. The HF filter is operable such that fluid that does not pass through a filter membrane can leave the HF filter through an outlet as retentate 212 to be reintroduced to the bioreactor 220. Permeate fluid can be pulled through the HF filter through the action of two or more pumps to enter two or more flowpaths. Under certain operating conditions, such as operating in a perfusion process, a first pump 206 is operable to pull a permeate 203 into a first flowpath 204 to form a perfusion permeate 207. The first pump 206 is operably in communication with a sensor 205 operable for accurate measurement of a fluid flow rate, such that the first pump 206 is operable for maintaining a fluid flow rate suitable for a perfusion process, for example, wherein a perfusion permeate 207 from the HF filter is pulled at a consistent and/or desirable rate. Under certain operating conditions, such as operating in a harvest process, a second pump 210 is operable to pull a permeate 203 into a second flowpath 208 to form a harvest permeate 211. The second pump 210 and harvest permeate 211 are optionally operable for communication with one or more sensors and/or fluid communication with one or more downstream filtering units and/or processing units suitable for maintaining a harvest process. In general, the second flowpath 208 and second pump 210 draw permeate at a higher fluid flow rate than a first flowpath 204 and a first pump 206.

IV.A. Bioreactors

In some embodiments, described herein are systems that comprise a multi-purpose assembly. In some embodiments, such a system is operable with a bioreactor. A bioreactor is a vessel operable for supporting a biologically active environment. A bioreactor may be suitable for supporting either an aerobic and/or an anaerobic biological activity. In some embodiments, a bioreactor may be a batch bioreactor, a fed batch bioreactor, a continuous bioreactor, a semi continuous bioreactor, or a perfusion bioreactor. In some embodiments, a bioreactor may be of any size. In some embodiments, a bioreactor may be suitable for cell growth and/or maintenance during any phase of cell culture (e.g., lag phase, log phase, stationary phase, death phase, etc.). In some embodiments, a bioreactor may be of any suitable material (e.g., stainless steel, etc.). In some embodiments, a bioreactor may have one or more input ports and/or one or more output ports. In some embodiments, a bioreactor may be operable for connection with more than one multi-purpose filter assembly as described herein. In some embodiments, wherein a bioreactor is operable with more than one multi-purpose filter assembly, such more than one multi-purpose filter assemblies may function simultaneously, or sequentially. In some embodiments, more than one bioreactor vessels can be utilized in tandem or sequentially.

In some embodiments, a bioreactor vessel can comprise a volume greater than or equal to about 0.25, 0.5, 0.75, 1, 1.5, 2, 2.5, 3, 3.5, 4, 4.5, 5, 10, 20, 30, 40, 50, 60, 70, 80, 90, 100, 200, 300, 400, 500, 600, 700, 800, 900, 1,000, 1,100, 1,200, 1,300, 1,400, 1,500, 1,600, 1,700, 1,800, 1,900, 2,000, 2,100, 2,200, 2,300, 2,400, 2,500, 2,600, 2,700, 2,800, 2,900, 3,000, 3,100, 3,200, 3,300, 3,400, 3,500, 3,600, 3,700, 3,800, 3,900, 4,000, 4,100, 4,200, 4,300, 4,400, 4,500, 4,600, 4,700, 4,800, 4,900, 5,000, 5,100, 5,200, 5,300, 5,400, 5,500, 5,600, 5,700, 5,800, 6,000, 6,100, 6,200, 6,300, 6,400, 6,500, 6,600, 6,700, 6,800, 6,900, or 7,000 liters. In some embodiments, a bioreactor vessel can comprise a volume less than or equal to about 15 liters. In some embodiments, a bioreactor vessel can comprise a volume less than or equal to about 50 liters. In some embodiments, a bioreactor vessel can comprise a volume greater than or equal to about 3,000 liters. In some embodiments, a bioreactor vessel can comprise a volume greater than or equal to about 6,000 liters.

In some embodiments, a bioreactor vessel total volume is larger than a working volume of cell culture fluid comprised in the bioreactor. In some embodiments, a bioreactor vessel total volume may be greater than or equal to about 10, 25, 50, 100, 500, 1,00, 2,000, 3,000, 4,000, 5,000, 6,000, 7,000, 8,000, 9,000, 10,000, 11,000, 12,000, 13,000, 14,000, 16,000, 17,000, 18,000, 19,000, 20,000 liters, or any range derivable therein.

In some embodiments, a bioreactor vessel is maintained at a set temperature. In some embodiments, a bioreactor vessel is maintained at about 20 to about 40° C., about 30 to about 38° C., or about 35 to about 38° C. In certain embodiments, a bioreactor vessel is maintained at about 20, 21, 22, 23, 24, 25, 26, 27, 28, 29, 30, 31, 32, 33, 34, 35, 36, 37, 38, 39, 40, 41, 42, or 43° C. In certain embodiments, a bioreactor vessel is maintained at about 37° C.

IV.B. Cell Culture Fluid

In some embodiments, described herein are systems and/or assemblies that may comprise cell culture fluid. In some embodiments, cell culture fluid may be any cell culture fluid known in the art. In some embodiments, cell culture fluid may comprise cells, biologics, cell culture media, metabolites, etc. In some embodiments, a feed stream of cell culture media is added to a cell culture fluid. In some embodiments, a cell culture media may comprise a concentrated amount of cell nutrients, such as sugars, peptides, vitamins, lipids, etc. In some embodiments, fresh cell culture media is introduced to a bioreactor as shown in FIG. 2 through the optional input port 223. In some embodiments, a permeate cell culture media comprises a lower concentration of metabolites and/or a higher concentration of biologics than cell culture fluid and/or fresh cell culture media. In some embodiments, a harvest permeate cell culture media comprises a higher concentration of biologics than a perfusion permeate cell culture media.

### V. Methods of Use

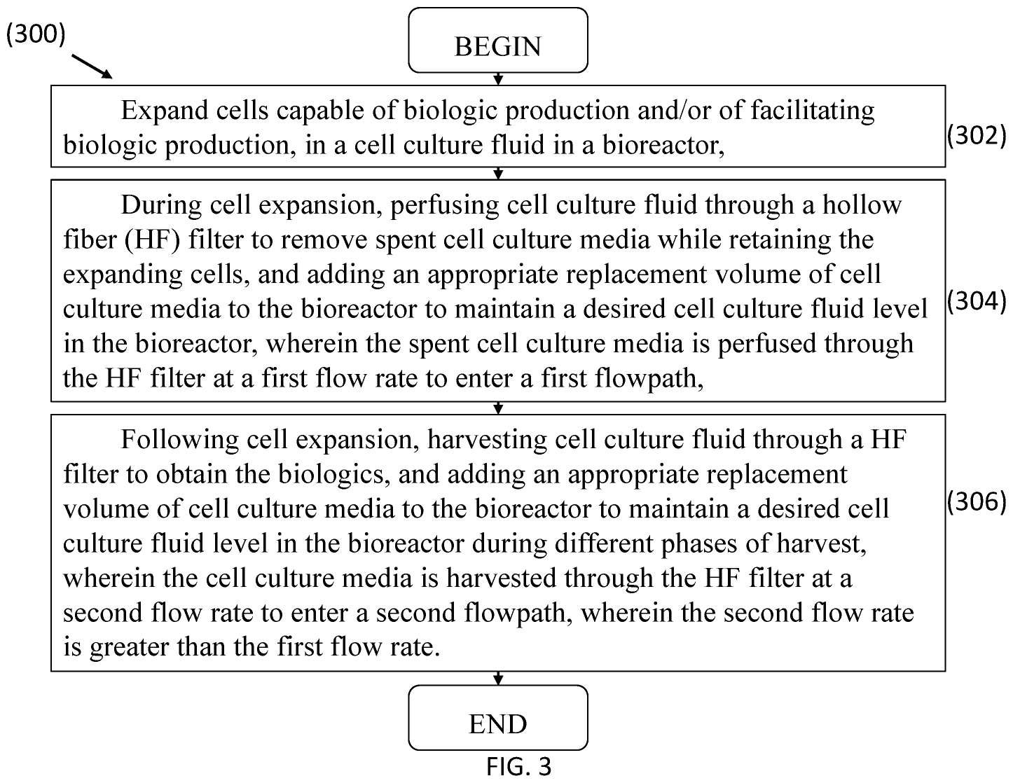

Referring to FIG. 3, illustrated is a non-limiting example flowchart illustrating a method for production of biologics using a HF filter capable of operating during both a perfusion and a harvest process. In certain embodiments, the HF filter is operable for tangential flow. In certain embodiments, the HF filter is a TFDF® filter. The method 300 can comprise, at step 302, expanding a population of cells capable of producing biologics and/or capable of facilitating the production of biologics (e.g., capable of expanding a virus population, acting as feeder cells, etc.) in a cell culture fluid in a bioreactor. During step 304, the cells are expanded using a perfusion process cell culturing technique, wherein cell culture fluid is passed through a HF filter to remove spent cell culture media (e.g., the media is perfused through the HF filter membrane), while cells are retained in the HF filter lumen and cycled through a retentate stream back to the bioreactor. During a perfusion stage, the cell culture spent media is perfused through the HF filter at a first flow rate to enter a first flowpath (e.g., a perfusion permeate flowpath). In certain embodiments, an appropriate replacement volume of cell culture media is added to the bioreactor to maintain a desired cell culture fluid level in the bioreactor. In certain embodiments, the replacement volume of cell culture media is about the same, less than, or greater than the volume of spent cell culture media that is removed. In certain embodiments, the replacement volume of cell culture media is fresh cell culture media. During step 306, following cell expansion using a perfusion process, a harvesting process is initiated. In certain embodiments, between the perfusion process and harvesting process, an intermediary process is performed. During such a harvesting process, cell culture media is perfused through the HF filter at a second flow rate to enter a second flowpath (e.g., a harvest permeate flowpath). In certain embodiments, an appropriate replacement volume of cell culture media is added to the bioreactor to maintain a desired cell culture fluid level in the bioreactor during different phases of harvest. In certain embodiments, the replacement volume of cell culture media is the about same, less than, or greater than the volume of the spent cell culture media that is removed. In certain embodiments, the replacement volume of cell culture media is fresh cell culture media. The second flow rate associated with the second flowpath is higher than the first flow rate associated with the first flowpath. In specific embodiments, a first flow rate in a first flowpath is less than or equal to about 5 LPM, while a second flow rate in a second flowpath is greater than about 5 LPM. In some embodiments, the method comprises the use of any systems or assemblies described herein.

In some embodiments, systems and apparatuses described herein can be utilized in various processing schemes, such as but not limited to: perfusion, batch, fed batch, semi-continuous processing (e.g., continuous harvest, extended harvest, etc.), and/or continuous processing.

In some embodiments, systems and apparatuses described herein are utilized in methods comprising high liquid volumes, high liquid flowrates, high flux, and/or high pressure, and are suitable for use with sensitive cells (e.g., animal cells, e.g., mammalian cells) that are prone to shearing at high liquid volumes, high liquid flowrates, high flux, and/or high pressure.

V.A. Cells

The assemblies and systems described herein can be used with cells of any type. In particular embodiments, cells are considered relatively sensitive cells (e.g., cells are shear sensitive, e.g., cells are prone to shearing at certain high volumes, flowrates, flux rates, and/or pressures) when compared to cells such as Escherichia coli or Saccharomyces cerevisiae. In particular embodiments, cells are animal cells. In particular embodiments, cells are mammalian cells. In certain embodiments, the cells are immune effector cells (e.g., lymphocytes). In certain embodiments, the cells are T-cells. In certain embodiments, the cells are B-cells. In certain embodiments, the cells are NK cells. In certain embodiments, the cells are stem cells. In certain embodiments, the cells are induced pluripotent stem cells (iPSCs). In certain embodiments, the cells are stem cells and/or iPSCs that have been differentiated into a hematopoietic lineage. In certain embodiments, the cells are hematopoietic progenitor cells. In certain embodiments, cells are Chinese Ovarian Hamster (CHO) cells. In certain embodiments, cells are Human embryonic kidney 293 (HEK293) cells. In certain embodiments, cells are human fibrosarcoma cells (e.g., HT-1080 cells). In certain embodiments, cells are derived from immortalized human embryonic cells (e.g., PER.C6 cells). In certain embodiments, cells are fusions of HEK293-S and a human B-cell line (e.g., HKB-11 cells). In certain embodiments, cells are derived from human hepatocellular carcinoma cells (e.g., HuH-7 cells). In certain embodiments, cells are feeder and/or host cells for the production of one or more viruses.

V.B. Products

The assemblies and systems described herein can be used to produce a product. In certain embodiments, a product is a cell (e.g., a stem cell, a cell of hematopoietic lineage, an immune effector cell, etc.). In certain embodiments, a product is a biologic. In certain embodiments, biologics may be of any type. In particular embodiments, biologics may be but are not limited to, small molecules, peptides, proteins, antibodies (including antibody derivatives such as but not limited to Fc fusions, scFvs, multispecific antibodies, bispecific antibodies, etc.), carbohydrates, lipids, viruses, virus like particles, viral proteins/peptides, extracellular particles (e.g., microvesicles, exosomes, etc.), vaccines, and/or nucleotides (e.g., DNA and/or RNA molecules).

In certain embodiments, a biologic comprises, consists essentially of, or consists of an antibody or a functional unit thereof (e.g., a single-chain variable fragment (scFv), a heavy chain, a light chain, a fragment crystallizable (Fc) domain, etc.). In certain embodiments, a biologic comprises, consists essentially of, or consists of a viral vector. In certain embodiments, a biologic comprises, consists essentially of, or consists of an adeno-associated virus (AAV). In certain embodiments, a biologic comprises, consists essentially of, or consists of a lentivirus. In certain embodiments, a biologic comprises, consists essentially of, or consists of an adenovirus. In certain embodiments, a biologic does not comprise, consist essentially of, or consist of an AAV, lentivirus, and/or adenovirus. In certain embodiments, a biologic comprises, consists essentially of, or consists of an RNA molecule, such as but not limited to messenger RNA (mRNA), small interfering RNA (siRNA), microRNA (miRNA), long non-coding RNA (lncRNA), and/or antisense oligonucleotide (ASO). In some embodiments, a biologic comprises, consists essentially of, or consists of an antigenic molecule (e.g., a molecule that can induce an immune response in a subject).

V.C. Automation

In some embodiments, assemblies and systems described herein and methods of using the same can be automated. In certain embodiments, automation comprises integration of one or more human machine interfaces (HMI) and a processing device. In some embodiments, one or more sensors can provide a processing device with real-time data which can be displayed on the HMI. In some embodiments, actions required for changing of a biomanufacturing process, e.g., perfusion process, in-between process, or harvest process, can be programmed into a processing device and controlled at the HMI.

V.D. Timing

In some embodiments, assemblies and systems described herein and methods of using the same can be for any period of time. In certain embodiments, a perfusion process comprising use of a multi-purpose assembly as described herein can be for equal to or greater than about 0.25, 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 days, or longer than 60 days, or any range derivable therein. In certain embodiments, a perfusion process is about 1, 2, 3, 4, 5, 6, 7, 8, 9, 10, or greater than 10 days, or any range derivable therein. In certain embodiments, a perfusion process is about 5 days. In certain embodiments, a perfusion process is about 6 days.

In certain embodiments, a HF filter is not exchanged between a perfusion process and a harvest process. In certain embodiments, a harvest process comprising use of a multi-purpose assembly as described herein can be for equal to or greater than about 0.25, 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 days, or longer than 60 days, or any range derivable therein. In certain embodiments, a perfusion process and a harvest process comprising use of a multi-purpose assembly as described herein can be for equal to or greater than about 0.25, 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 days, or longer than 70 days, or any range derivable therein. In certain embodiments, a harvest process is about 2 days, about 3 days, about 4 days, about 5 days, about 6 days, about 7 days, about 8 days, about 9 days, about 10 days, or greater than about 10 days. In some embodiments, a harvest process is about 6 days. In some embodiments, a harvest process is longer than about 6 days. In some embodiments, a harvest process is a continuous harvest process.

### VI. Representative Experimental Results

VI.A. Systems Comprising a Multi-Purpose Assembly

Multi-purpose assemblies and systems comprising the components depicted in FIG. 1 and FIG. 2 were produced. Biomanufacturing trials were conducted for at least 10 different biologics, including at least 4 trials comprising combination perfusion and continuous harvest processes. Exemplary multi-purpose assemblies and systems comprising the same achieved filter flux levels up to 600 LMH, VVD rates of up to 4.3, VCD rates of up to 158×10⁶, and filter throughput rates of up to 57,500 L/m².

TABLE 2
Exemplary Trials, Results Summary

| | | Feedstock and Filter Information | | | | | | | |
|---|---|---|---|---|---|---|---|---|---|
| | | | Filter | | Test Outcome | | | | |
| | | | Surface | Processing | | | Final | Filter | |
| | | | Area | Volume | Flux | Max | VCD | throughput | |
| Site | Run | Biologic | (cm\hat{ }2) | (L) | (LMH) | VVD | (×10\hat{ }6) | (L/m2) | Processes |
| 1 | 1 | A | 150 | 19 | 105.56 | 2 | 60 | 24320 | P + CH |
| 1 | 2 | B | 150 | 30 | 191.67 | 2.3 | 70 | 28240 | P + CH |
| 2 | 3 | C | 1500 | 110 | 25.67 | 0.84 | 55 | 3192 | P + CH |
| 2 | 4 | D | 1500 | 51 | 21.25 | 1.5 | 55 | 2037 | P + CH |
| 1 | 5 | F | 30 | 10 | 600 | 4.3 | 140 | 40000 | P |
| 1 | 6 | G | 150 | 50 | 320 | 2.5 | 60 | 22,000 | P |
| 1 | 7 | H | 30 | 12.5 | 399 | 2.3 | 91.8 | 33,970 | P |
| 2 | 8 | I | 150 | 81 | 450 | 2 | 100 | 57,500 | P |
| 2 | 9 | J | 30 | 9.9 | 550 | 4 | 158 | 32,010 | P |
| 1 | 10 | A | 1500 | 450 | 423 | 3.5 | 88.1 | 38,667 | P |
| 1 | 11 | H | 450 | 113 | 398 | 3.8 | 76.4 | 39,675 | P |
| 1 | 12 | K | 750 | 188 | 398 | 3.8 | 78.3 | 30,080 | P |

KEY: Perfusion = P; Continuous Harvest = CH; Biologics are coded; Test sites are coded.

### VII. Additional Considerations

Any headers and/or subheaders between sections and subsections of this document are included solely for the purpose of improving readability and do not imply that features cannot be combined across sections and subsection. Accordingly, sections and subsections do not describe separate embodiments.

While the present teachings are described in conjunction with various embodiments, it is not intended that the present teachings be limited to such embodiments. On the contrary, the present teachings encompass various alternatives, modifications, and equivalents, as will be appreciated by those of skill in the art. The present description provides preferred exemplary embodiments, and is not intended to limit the scope, applicability, or configuration of the disclosure. Rather, the present description of the preferred exemplary embodiments will provide those skilled in the art with an enabling description for implementing various embodiments.

It is understood that various changes may be made in the function and arrangement of elements without departing from the spirit and scope as set forth in the appended claims. Thus, such modifications and variations are considered to be within the scope set forth in the appended claims. Further, the terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed.

In describing the various embodiments, the specification may have presented a method and/or process as a particular sequence of steps. However, to the extent that the method or process does not rely on the particular order of steps set forth herein, the method or process should not be limited to the particular sequence of steps described, and one skilled in the art can readily appreciate that the sequences may be varied and still remain within the spirit and scope of the various embodiments.

Some embodiments of the present disclosure include a system including one or more data processors. In some embodiments, the system includes a non-transitory computer readable storage medium containing instructions which, when executed on the one or more data processors, cause the one or more data processors to perform part or all of one or more methods and/or part or all of one or more processes disclosed herein. Some embodiments of the present disclosure include a computer-program product tangibly embodied in a non-transitory machine-readable storage medium, including instructions configured to cause one or more data processors to perform part or all of one or more methods and/or part or all of one or more processes disclosed herein.

Specific details are given in the present description to provide an understanding of the embodiments. However, it is understood that the embodiments may be practiced without these specific details. For example, circuits, systems, networks, processes, and other components may be shown as components in block diagram form in order not to obscure the embodiments in unnecessary detail. In other instances, well-known circuits, processes, algorithms, structures, and techniques may be shown without unnecessary detail in order to avoid obscuring the embodiments.

What is claimed is:

1. A bioreactor system, comprising:
a) a bioreactor including an input port and an output port;
b) a feed stream conduit operably connected to the output port and inlet of a Hollow Fiber (HF) filter, the feed stream conduit operable to carry fluid from the bioreactor to the HF filter;
c) a retentate stream conduit operably connected to the input port of the bioreactor and an outlet of the HF filter, the retentate stream conduit operable to carry fluid to the bioreactor from the HF filter; and
d) a multi-purpose assembly operably connected to the HF filter, the multi-purpose assembly including a first flow path and a second flowpath:
wherein the first flowpath includes a first pump operable to draw fluid from the HF filter and a flow meter operable to measure flow rate of fluid in the first flowpath;
wherein the second flowpath includes a second pump operable to draw fluid from the HF filter; and
wherein the first pump and the second pump are configured to have different pump capacities.

2. The bioreactor system of claim 1, wherein the HF filter is operable for tangential flow.

3.-4. (canceled)

5. The bioreactor system of claim 1, further including:
one or more clarification filter(s) in the first and/or second flow path.

6.-8. (canceled)

9. The bioreactor system of claim 1, wherein the retentate stream conduit is operable to carry cell culture fluid to the bioreactor from the HF filter,
wherein the first flowpath including the first pump operable to draw fluid from the HF filter, is operable to draw a perfusion permeate stream fluid from the HF filter, and
wherein the second flowpath including the second pump operable to draw fluid from the HF filter, is operable to draw a harvest permeate stream fluid from the HF filter.

10.-13. (canceled)

14. The bioreactor system of claim 1, wherein the first pump is operable to draw fluid from the HF filter at a flow rate of between about 0.01 liters per min (LPM) and 5 LPM.

15.-16. (canceled)

17. The bioreactor system of claim 1, wherein the first flowpath and pump are operable to draw a feed flow rate from the HF filter of about 0.8 to about 2.2 liters per fiber per minute (L/fiber/min).

18.-19. (canceled)

20. The bioreactor system of claim 1, wherein the first pump is operable to draw fluid from the HF filter while maintaining a VCD of greater than about 25×10⁶ cells/mL.

21. The bioreactor system of claim 1, wherein the first pump is operable to draw fluid from the HF filter at a rate of between about 0.1 and about 5 bioreactor vessel volumes per day (VVD).

22. (canceled)

23. The bioreactor system of claim 1, wherein the first pump is operable to draw fluid from the HF filter at a filter flux rate of between about 50 and about 800 liters per square meter per hour (LMH).

24.-27. (canceled)

28. The bioreactor system of claim 1, wherein the first pump is operable to draw fluid from the HF filter at a throughput equal to between about 10,000 liters per square meter (L/m²) to about 70,000 L/m².

29. The bioreactor system of claim 1, wherein the first pump is operable to draw fluid from the HF filter while maintaining a shear rate (s−1) of less than about 5,000 s−1.

30.-40. (canceled)

41. The bioreactor system of claim 1, wherein the bioreactor system is capable of operating in a perfusion process, an in-between perfusion and harvest process, and a harvest process.

42.-43. (canceled)

44. The bioreactor system of claim 1, wherein the bioreactor system is capable of operating in a continuous harvest process.

45. The bioreactor system of claim 1, further comprising a human machine interface (HMI) control unit, and wherein the HMI control unit is programmed and can display perfusion process controls, in-between perfusion and harvest process controls, or harvest process controls.

46.-51. (canceled)

52. The bioreactor system of claim 1, further comprising a second flow meter operable to measure flow rate of fluid in the second flowpath and accurately monitoring a flow rate of about 0 to about 10 LPM.

53. (canceled)

54. The bioreactor system of claim 1, wherein the second pump is operable to draw fluid from the HF filter at a throughput equal to between about 10,000 liters per square meter (L/m²) to about 70,000 L/m².

55. The bioreactor system of claim 1, wherein the second pump is operable to draw fluid from the HF filter at a filter flux rate of between about 150 and about 900 liters per square meter per hour (LMH).

56. (canceled)

57. The bioreactor system of claim 1, wherein the second flowpath and pump are operable to draw a feed flow rate from the HF filter of about 1 to about 3 liters per fiber per minute (L/fiber/min).

58.-59. (canceled)

60. The bioreactor system of claim 1, wherein the second pump is operable to draw fluid from the HF filter at a flow rate of between about 0.01 LPM to about 18 LPM.

61.-64. (canceled)

65. The bioreactor system of claim 1, wherein the first flowpath has an accuracy requirement of about 1% and/or wherein the second flowpath has an accuracy requirement of about 3%.

66.-67. (canceled)

68. The bioreactor system of claim 1, wherein the HF filter comprises isotropic pore structures and/or pores with an average lumen diameter of about 0.65 μm to about 8 μm.

69. The bioreactor system of claim 1, operable for use with shear sensitive cells and/or operable for use with cells for the production of a biologic.

70.-75. (canceled)

76. A multi-purpose filter assembly, comprising:
a) a Hollow Fiber (HF) filter; and
b) a multi-purpose assembly operably connected to the HF filter, the multi-purpose assembly including a first flow path and a second flowpath:
wherein the first flowpath includes a first pump operable to draw fluids from the HF filter and a flow meter operable to measure flow rate of fluid in the first flowpath;
wherein the second flowpath includes a second pump operable to draw fluid from the HF filter; and
wherein the first pump and the second pump are capable of having different pump capacities and accuracy ratings.

77.-131. (canceled)

132. A method of producing biologics from cells, comprising:
a) expanding cells in a cell culture fluid in a bioreactor,
b) during cell expansion, perfusing cell culture fluid through a Hollow Fiber (HF) filter to remove spent cell culture media while retaining the expanding cells, and adding an appropriate replacement volume of cell culture media to the bioreactor to maintain a desired cell culture fluid level in the bioreactor, wherein the spent cell culture media is perfused through the HF filter at a first flow rate to enter a first flowpath, and
c) following cell expansion, harvesting cell culture fluid through a HF filter to obtain the biologics, and adding an appropriate replacement volume of cell culture media to the bioreactor to maintain a desired cell culture fluid level in the bioreactor during different phases of harvest, wherein the cell culture media is harvested through the HF filter at a second flow rate to enter a second flowpath, wherein the second flow rate is greater than the first flow rate.

133.-158. (canceled)
